SEQUENCE LISTING (4 sequences)

SEQ ID NO: 1 - DNA, 35 bp, Unknown
  Description of Unknown Illustrative oligonucleotide of the invention
gtccaaagtcaggtcacagtgacctgatcaaagtt

SEQ ID NO: 2 - DNA, 32 bp, Unknown
  Description of Unknown Illustrative oligonucleotide of the invention
tccaaagtcaggtcacagtgacctgatcaaag

SEQ ID NO: 3 - DNA, 40 bp, Artificial Sequence
  Description of Artificial Sequence Synthetic oligonucleotide
gttacaaggtctaaacccgagttacaaggtctaaacccga

SEQ ID NO: 4 - DNA, 32 bp, Artificial Sequence
  Description of Artificial Sequence Synthetic oligonucleotide
tccaaagtcatatcacagtgggctgatcaaag

(19) United States
(12) Patent Application Publication
(10) Pub. No.: US 2009/0062130 A1
(43) Pub. Date: Mar. 5, 2009

(54) NUCLEOSOME-BASED BIOSENSOR

(75) Inventors: Richard Arthur Steinman, Pittsburgh, PA (US); Sanford H. Leuba, Pittsburgh, PA (US)

Correspondence Address: FOLEY AND LARDNER LLP;SUITE 500, 3000 K STREET NW, WASHINGTON, DC, 20007, US

(73) Assignee: UNIVERSITY OF PITTSBURGH - OF THE COMMONWEALTH SYSTEM OF HIGHER EDUCATION

(21) Appl. No.: 11/687,859

(22) Filed: Mar. 19, 2007

Related U.S. Application Data
(60) Provisional application No. 60/783,400, filed on Mar. 20, 2006.

(51) Int. Cl. C40B 30/00; C12Q 1/68; C40B 40/06; C40B 50/08

(52) U.S. Cl. 506/7; 435/6; 506/16; 506/27

(57) ABSTRACT

A nucleosome-based biosensor can detect events of transcriptional activity. The biosensor includes a nucleosome-forming DNA that contains at least one nuclear responsive DNA sequence; a core histone octamer and at least two labels. In one version, the two labels are placed on the DNA. In another version, one of the labels is attached to the DNA, while the other to the core histone octamer. The sensor function is effected by measuring an emission signal, associated with the labels, which is sensitive to whether the nucleosome-forming DNA is or is not in nucleosomal configuration. The biosensor finds application, for example, in high-throughput screening for ligands to known and orphan nuclear receptors, respectively.

### CROSS-REFERENCE TO RELATED APPLICATIONS

The present application claims priority to U.S. provisional patent application No. 60/783,400, filed Mar. 20, 2006.

### BACKGROUND OF THE INVENTION

Nuclear receptors (NRs) modulate transcription, by binding small, lipophilic ligands, and have a profound impact on normal cellular function and on development. Aberrant NR function occurs frequently in cancers. See, e.g., Sharif and Privalsky Cell 66: 885-93 (1991); de The et al., loc. cit.: 675-84. This observation has prompted the development of therapeutic ligands that target steroid hormone and retinoid receptors, e.g., tamoxifen in breast cancer and ATRA in promyelocytic leukemia.

For a large number of so-called “orphan” nuclear receptors, however, natural ligands or artificial modulators have yet to be identified. Accordingly, characterizing ligands for orphan nuclear receptors could illuminate new approaches to controlling cellular behavior and disease processes, such as cancer.

In relation to cancer specifically, there are compelling reasons to seek novel nuclear receptor ligands. Ligands directed against the same receptor can exhibit different tissue-specific responses and, hence, have different side effects in therapeutic use. Thus, tamoxifen is an estrogen receptor (ER) antagonist in breast tissue and an agonist in uterine tissue, whereas the ER antagonist raloxifene lacks agonist activity in uterus. See, e.g., Grese et al., Proc. Nat'l Acad. Sci. USA 94: 14105-110 (1997). Also, the ongoing identification of ligands for nuclear receptors uncovers novel physiological functions of nuclear receptors or their ligands. For instance, the identification of 1,1-bis(3-indolyl)-1-(p-anisyl)methane as an activating ligand of the nuclear hormone Nur77 demonstrated an anticancer effect of Nur77 activation in pancreatic cancer cells. See Chintharlapalli et al., J. Biol. Chem. 280: 24903-914 (2005).

Current screens for nuclear receptor ligands, including biochemical assays, usually focus on ligand-receptor binding strictly. Yet ligand-receptor binding per se does not provide information on the transcription activity of the ligand. Consequently, a need exists for an alternative ligand screening methodology.

### SUMMARY OF THE INVENTION

The present invention provides a nucleosome-based biosensor and related methodology for making and using it, for example, in detecting transcriptional activity. According to one embodiment, the biosensor comprises (1) at least one surface with which at least one nucleosome is associated, each nucleosome thereof comprising a nucleosome-forming DNA that comprises at least one transcription regulating DNA sequence element and that is labeled with a first label and a second label, such that the first label and the second label are (a) in a first proximity when the nucleosome-forming DNA is in nucleosomal configuration and (b) in a second proximity, differing from the first proximity, when the DNA is not in nucleosomal configuration, and (2) a detector for the emission signal correlated with the labels, wherein the aforementioned surface is in an operative relationship to said detector such that the emission signal reaches the detector.

According to another embodiment, the biosensor comprises (A) at least one surface with which at least one nucleosome is associated, each nucleosome thereof comprising (i) a nucleosome-forming DNA that is comprised of at least one nuclear hormone response DNA sequence element and that is labeled with at least one first label and (ii) a core histone octamer that is labeled with at least one second label, and (B) a detector for an emission signal correlated with said labels, wherein the surface is in an operative relationship to the detector, as described above.

Yet another aspect of the invention is a transcriptional chip, comprising (1) a substrate and, attached thereto, (2) a plurality of nucleosomes, each nucleosome thereof comprising a nucleosome-forming DNA that comprises at least one transcription regulating DNA sequence element and that is labeled with a first label and a second label, such that the first label and the second label are (a) in a first proximity when the nucleosome-forming DNA is in nucleosomal configuration and (b) in a second proximity, differing from the first proximity, when the DNA is not in nucleosomal configuration.

Another transcription chip, representing a further aspect of the invention, comprises (A) a substrate and, attached thereto, (B) a plurality of nucleosomes, each comprising (i) a nucleosome-forming DNA that comprises at least one transcription regulating sequence element and is labeled with at least one first label and (ii) a core histone octamer that is labeled with at least one second label.

The invention also provides a method for making a transcriptional chip, comprising (1) disposing on a substrate a plurality of nucleosome-forming DNAs, wherein each DNA of the plurality comprises at least one transcription regulating DNA sequence element and is labeled with a first label and a second label, such that the first label and the second label are (a) in a first proximity when the nucleosome-forming DNA is in nucleosomal configuration and (b) in a second proximity, differing from the first proximity, when the DNA is not in nucleosomal configuration, and (2) bringing said DNAs into contact with a plurality of core histones under nucleosome-forming conditions. In some embodiments, the at least one transcription regulating element can be at least one nuclear responsive DNA sequence element.

Alternatively, a method of making a transcription chip comprises (A) disposing on a substrate a plurality of nucleosome-forming DNAs, wherein each DNA of said plurality comprises at least one transcription regulating DNA element and is labeled with at least one first label (B) bringing said DNAs into contact with a plurality of core histone octamers, wherein each core histone octamer of said plurality is labeled with at least one second label, under nucleosome-forming conditions.

Another aspect of the invention relates to determining activity of putative ligand towards a nuclear receptor. An inventive method to this end comprises (1) providing at least one nucleosome comprising (i) a nucleosome-forming DNA that comprises at least one nuclear hormone response DNA sequence element of the nuclear receptor and that is labeled with a first label and a second label, such that the first label and the second label are (a) in a first proximity when the nucleosome-forming DNA is in nucleosomal configuration and (b) in a second proximity, differing from the first, when the DNA is not in nucleosomal configuration, (2) exposing said nucleosome to at least one putative ligand, and (3) measuring for a change in emission signal, associated with the labels, that is consequent to the aforementioned exposing. Along the lines indicated above, an alternative embodiment of this approach involves the use of a nucleosome that comprises (i) a nucleosome-forming DNA that comprises at least one nuclear hormone response DNA sequence element for said nuclear receptor and that is labeled with at least one first label and (ii) a core histone octamer that is labeled with at least one second label. Again, upon exposure of the nucleosome to at least one putative ligand, one measures for a change in emission signal, associated with said labels, that is consequent to the exposing. In another embodiment, step (B) involves exposure of the nucleosome(s) to a composition comprised of a transcriptional activator and nuclear extracts from cells of a tissue.

Although the foregoing refers to particular preferred embodiments, the present invention is not so limited. The knowledgeable reader will apprehend that various modifications may be made to the embodiments explicitly disclosed here, and that such modifications are intended to be within the scope of the present invention.

### BRIEF DESCRIPTION OF THE DRAWINGS

FIG. 1(A) illustrates a concept of fluorescence resonance energy transfer, or FRET, as applied in the present invention; (B) schematically depicts scanning confocal fluorescence microscope or SCFM, and (C) provides a photograph of SCFM.

FIGS. 2(A-C) present an example of SCFM data and data analysis for a four-way junction (4WJ) DNA substrate: (A) Schematic of transitions of a doubly-labeled four-way junction. The two diagrams indicate the four-way junction DNA in a high-FRET state where the acceptor and donor dyes are in close proximity, and in a low-FRET state where the two dyes are much further apart. (B) Behavior of a single four-way junction (4WJ) DNA in 50 mM NaCl, 50 mM MgCl₂, 10 mM Tris-HCl, 0.5 mM EDTA, pH 7.5: the time trajectories for the intensities of the donor and acceptor dyes have been overlaid and show anti-correlated behavior indicative of FRET. Periods of high intensity acceptor signal (red) indicate the high FRET state; as acceptor intensity drops, the donor intensity (green) increases and indicates the low FRET state. (C) Dwell time analysis of a single 4WJ DNA molecule in high-FRET state (upper panel) and low-FRET state (lower panel).

FIGS. 3(A-C) demonstrate a fluorescent behavior of naked DNA and nucleosomes formed on the 164 bp GUB nucleosome-forming sequence labeled with Cy3 and Cy5. Each image is one video-frame (inverted contrast) from the ICCD camera and is split into two parts, showing Cy3 fluorescence on the left side and Cy5 fluorescence on the right side, respectively. Each black dot represents the fluorescence emitted from a single dye. The Gaussian peak maxima±the standard deviation are listed in each histogram. (A) Naked DNA in 50 mM NaCl, 10 mM Tris-HCl, pH 7.5, in the presence of oxygen scavenger system. Individual Cy3 signals (black spots) seen on left panel; very few Cy5 signals on right panel. (B) Nucleosomes were reconstituted in solution from DNA and core histones by the salt-jump method (51). Assembled nucleosomes were attached to the surface, rinsed and imaged in 5 mM, 50 mM, or 250 mM NaCl. The Gaussian peak parameters and numbers of data points for the different salt concentrations are: 0.87±0.20 (n=657) for 5 mM NaCl, 0.88±0.22 (n=636) for 50 mM NaCl, and 0.88±0.21 (n=701) for 250 mM NaCl. The data for the three salt concentrations are practically indistinguishable and, hence, are presented as a single, combined peak. (C) Total dissociation of histone octamers in (B) upon washing with 2 M NaCl to return to naked DNA conformation. The numbers of data points used for the distribution histograms are as follows: (A) 461, B) 1994, and (C) 472. Data from (49).

FIG. 4 illustrates a flow sorting biosensor system according to the present invention and the use of such a system to isolate a functional ligand from a mixture of putative ligands.

FIG. 5 illustrates an operation of a nucleosome-based biosensor according to one of the embodiments of the invention.

FIG. 6(A) depicts the operation of a biosensor of the invention, for determining a functional significance of a polymorphism in a transcription-regulating DNA sequence element. FIG. 6(B) similarly illustrates an inventive biosensor for determining the transcriptional activity of a putative ligand.

FIG. 7(A) depicts a conserved modular structure of a nuclear receptor. FIG. 7(B) illustrates binding of estrogen receptor to an estrogen receptor DNA element.

FIGS. 8(A)-(C) illustrate an assembly of nucleosome forming DNA containing estrogen receptor DNA sequence element (ERE) into nucleosomes. FIG. 8(A) shows 147 base pair long double-stranded DNA having Cy5 (red mark) and TAMRA (green mark) fluorophore labels, estrogen receptor DNA sequence element (blue line) and biotin (yellow). FIG. 8(B) shows a model of the nucleosome forming DNA containing ERE assembled into a nucleosome based on GUB pseudodyad position. ERE is shown as thick green double helix. Histone octomer helices are shown as ribbons. Cy5, TAMRA labels and Biotin are indicated. FIG. 8(C) represents results of FRET study of a nucleosome formed by the nucleosome-forming DNA containing ERE. Images were acquired at 10 ms over 30 seconds. Cy5/TAMRA intensity is shown in arbitrary units; Cy5 fluorescence is shown in magenta, TAMRA in blue. Heightened Cy5 signal over first 28 seconds is compatible with FRET and supports assembly of the ERE-DNA into a closed nucleosomal conformation. Nucleosomes were assembled using the salt jump method. They were attached to the quatz slide and then TAMRA-Cy5 signals were recorded using the confocal scanning fluorescent microscope.

### DETAILED DESCRIPTION

As noted, the present invention provides a nucleosome-based biosensor, as well as methodology for making and using the biosensor, particularly in the context of detecting transcriptional activity and identifying inducers affecting such activity. A central component of the inventive biosensor is a nucleosome-forming DNA that contains at least one transcription regulating DNA sequence.

In one biosensor configuration, the DNA is tagged with two labels, while the histone octamer is unlabeled. Another configuration is characterized by a placement of the labels on both nucleosome-forming DNA and nucleosome histone octamer. Pursuant to a third configuration, labels are associated only with histone proteins, while the DNA is not tagged.

With an appropriate detector, as discussed below, a biosensor of the invention can monitor the dynamic state of a single nucleosome or a population of nucleosomes, by measuring an emission signal associated with the labels.

### Nucleosomes

In this description the term “nucleosome” denotes a DNA-protein complex comprised of a core particle of 1.6 left-handed turns of DNA (roughly, 147 base pairs) wound around a protein complex, the histone octamer: a mononucleosome. The histone octamer is a set of eight basic proteins, which are among the most well-conserved of eukaryotic proteins. The histone octamer comprises a central tetramer, (H3/H4)₂, flanked by H2A/H2B dimers. The structure of a single histone molecule includes three major α helices with positively-charged loops protruding at the N-terminals. The nucleosome must undergo certain conformational changes to allow processes that require access to the DNA template.

### Nucleosome-Forming DNA

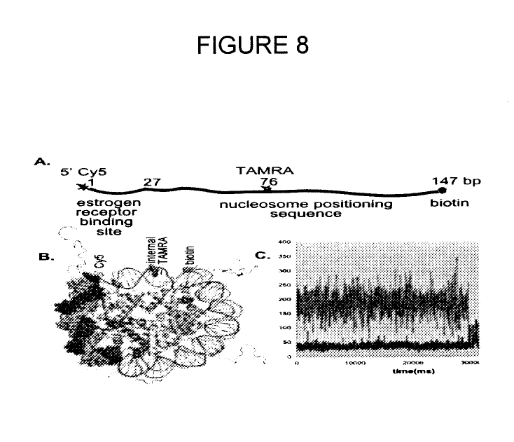

The phrase “nucleosome-forming DNA” denotes any DNA capable of binding to histone octamer to form a nucleosome. The ability of DNA sequence to bind to histone octamer can be tested in a digestion assay with micrococcal nuclease (MNase) or methidium EDTA II (MPE), as described, for example, in Karymov et al., FASEB J 15: 2631-41 (2001), Tomschik et al., Struct. Fold. Des. 9: 1201-11 (2001); Leuba et al., Proc. Nat'l Acad. Sci. USA 100: 495-500 (2003), and Tomschik et al., loc. cit. 102: 3278-83 (2005). In addition, the ability of a DNA sequence to reconstitute nucleosome can be revealed by nucleosome gel shifts in nondenaturating gels. See Tomschik et al. (2001), supra, particularly FIGS. 8A and 8C and related text. The nucleosome-forming DNA can include the core DNA of a nucleosome that wraps around the histone octamer. The nucleosome-forming DNA can further comprise “linker DNA”, i.e. DNA adjacent to the core DNA. In contrast to core DNA, linker DNA can vary in length from 8 to 200 base pairs.

The nucleosome-forming DNA can be, for example, a synthetic DNA sequence that is prepared using a known nucleosomal DNA sequence as a template, via conventional techniques, such polymerase chain reaction (PCR). Illustrative of such templates is the GUB sequence, see, e.g., Tomschik et al., Proc. Nat'l Acad. Sci. USA 102: 3279 (2005), the nucleosome B sequence of mouse mammary tumor virus, the 5S rDNA sequence, described by An et al., Proc. Nat'l Acad. Sci. USA 95: 3396-401 (1998), and the 200-bp “601” sequence generated by an in vitro physical selection (SELEX), as described by Lowary and Widom, J. Mol. Biol. 276: 19-42 (1998), and Thastrom et al., loc. cit. 288: 213-29 (1999).

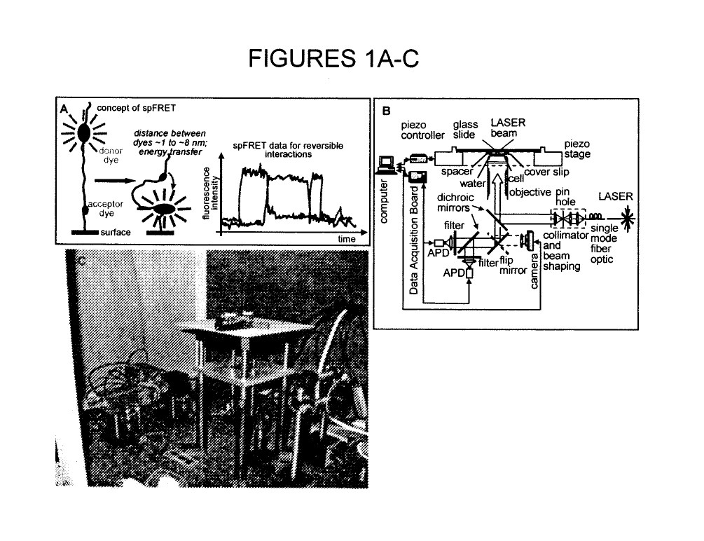

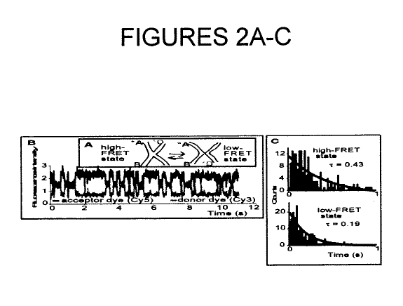

The phrase “nucleosomal configuration” signifies the arrangement of nucleosome-forming DNA wrapped around the core histone octamer, forming a nucleosome. Conversely, “non-nucleosomal configuration” connotes a state in which nucleosome-forming DNA is not wrapped around the core histone octamer (“naked DNA”). At the molecular level, these characterizations are relative, in the sense that nucleosome-site exposure entails a unwrapping and rewrapping of DNA that proceeds, in a finite time frame, according to particular kinetics. See Li et al. (2005), supra. At the operational level, however, for a biosensor device of the present invention the transition from one configuration to the other is essentially digital (+/−) for a single nucleosome, in keeping with the sensitivity level of the device in question. See FIGS. 1A and 2B, for instance.

### Nucleosome Assembly/Disassembly

Nucleosome-forming conditions can be effected by salt dialysis/salt jump, as described, for example, by Tatchell and van Holde, Biochemistry 5296 (1977), and Tomschik et al. (2005), supra, using an approximate equal weight of DNA to histone in 10 mM Tris-HCl, pH 7.5, 0.5 mM EDTA, [TE] 2 M NaCl that is stepwise diluted to 1, 0.75, and 0.5 M NaCl with TE, incubating at 37° C. for 20 minutes between each dilution. Thereafter, the nucleosomes thus formed can be dialyzed versus TE overnight. Alternatively, nucleosomes can be formed by incubating DNA with core histones (total histone concentration 0.1 mg/ml) and histone chaperone nucleosome assembly protein 1 (NAP-1) (0.2 mg/ml in 150 mM NaCl/10 mM Tris-HCl, pH 8.0/1 mM EDTA), incubated at 37° C. for 30 minutes, as described in Leuba et al., Proc. Nat'l Acad. Sci. USA 100: 495-500 (2003).

Conditions also are known that are conducive to nucleosome disassembly. Such conditions include those that are favorable to partial unwrapping of DNA from the histones, such as passage of an RNA polymerase, see CHROMATIN STRUCTURE AND DYNAMICS: STATE-OF-THE-ART, Zlatanova, J. and Leuba, S. H. (eds.), Elsevier (2004), at 479-80, and those that promote complete unwrapping of the DNA, such as 2 M NaCl. See Leuba et al. (2003), supra, at. 495. Thus, treatment of a nucleosome with 2 M NaCl results in complete histone disassembly.

### Fluorescent Labels

The labels can be fluorescent labels. In some embodiments, the first and second labels can be fluorescent labels which constitute a donor-acceptor pair. Measurement of an emission signal for a donor-acceptor pair entails illuminating the biosensor with a light with a wavelength exciting the donor. Preferably, the excitation light is a monochromatic light from a laser source. The biosensor detects the dynamic state of the nucleosome by measuring fluorescence intensities at the respective emission wavelengths of donor and acceptor or the ratio of such intensities.

Fluorescent labels forming a donor-acceptor pair are placed on the nucleosome so that the distance between the labels, when the DNA is unwrapped or “naked,” i.e., when the labels are in the first proximity, is greater that the distance between the labels when the DNA is wound around histones to form a nucleosome, i.e., when the labels are in the second proximity. In the context of fluorescent donor-acceptor labels, when the labels are in the second proximity the distance between the donor and acceptor typically is less than about 10 nm, which is the maximum Förster radius, and preferably less than about 5 nm and, more preferably, less than about 3 nm. Operationally, the incidence of the second proximity means that the FRET signal is observed, i.e., fluorescence at the acceptor emission wavelength occurs upon illumination of the DNA at the excitation wave length of the donor. First proximity pertains when the distance between donor and acceptor is greater than the Förster radius for the particular donor-acceptor pair. Thus, when the labels are in the first proximity the FRET signal is not observed at all or is substantially less than the FRET signal for the donor-acceptor pair in the interfering proximity.

If the absorption bands of the donor and acceptor are overlapping, a correction for the direct acceptor excitation should be made, in accordance with conventional practice. The fluorescent labels can be organic dyes. Illustrative of suitably paired organic dyes in this regard are Cy3 with Cy5, Cy5.5 with Cy 7, and tetramethylrhodamine (TMR or TAMRA) with Cy5. Conventional techniques are available for incorporating fluorescent dyes into nucleic acid sequences and attaching the dyes to proteins. See, e.g., Tomschik et al. (2005), supra; Li et al., Nature Structural & Molecular Biology 11: 763-69 (2004); Li et al., loc. cit. 12: 46-53 (2005). Measuring an emission signal from fluorescent labels, pursuant to the present invention, can be carried out by means of any instrumentation that allows following changes in fluorescence intensities rapidly over time. Examples of such apparatus are a wide-field, evanescent field fluorescence microscope (EFFM), as discussed, for instance, in Tomschik et al. (2005), supra, and Zheng HC, et al., Evanescent field fluorescence microscopy for analysis of protein/DNA interactions at the single-molecule level. In: PROTEIN-PROTEIN INTERACTIONS, A MOLECULAR CLONING MANUAL (2nd ed.) 1-16, Cold Spring Harbor Laboratory Press, 2005, and a smaller field laser confocal fluorescence microscope (SFCM), described in greater detail below. SCFM is preferred for fluorescent measurements of a one-molecule at a-time, and EFFM for population averaging fluorescent measurements.

### Two Fluorescent Labels on DNA

In one configuration, the biosensor has two fluorescent labels forming a donor acceptor pair attached to the nucleosome-forming DNA, while the histone octamer is unlabeled. Fluorescent labels can be placed on the same strand of the DNA. Alternatively, fluorescent labels can be on opposing strands of the DNA. Fluorescent labels can be placed on core DNA with a spacing of at least 30 base pairs. Thus, the labels can be at least 50 or at least 70 base pairs apart. Preferably, the fluorescent labels are placed on the core DNA approximately 75 pairs apart. When the DNA is prepared using a known nucleosomal DNA sequence as a template, one can use the crystallographic model of the nucleosomal DNA to select optimal positions for the fluorescent labels on the DNA. See, e.g., Tomschik et al. (2005), supra. In this situation, the labels can be positioned equidistant from the nucleosomal dyad position.

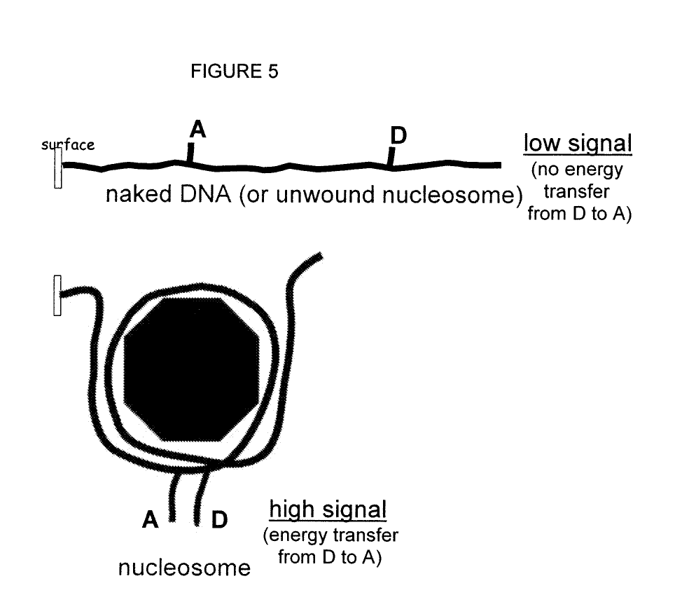

FIG. 5 illustrates a configuration of the biosensor that has two fluorescent labels, donor (D) and acceptor (A), on the nucleosome-forming DNA. On the top panel, the DNA is in non-nucleosomal configuration and D and A are separated, so that there is no energy transfer from D to A. On the bottom panel, the DNA is in nucleosomal configuration, i.e. the DNA is wrapped around a histone octamer depicted schematically in FIG. 5 as an octahedron, and D and A are in a close proximity allowing for an energy transfer from D to A. FIG. 5 depicts the DNA in non-nucleosomal configuration as completely unwrapped for illustrative purposes.

### Labels on Both DNA and Histones

The biosensor can also have labels placed for on both nucleosome-forming DNA and histone octamer. In this configuration, one or more donors are attached to one entity, i.e., DNA or histone octamer, while one or more matching acceptors are placed on the opposite entity. The number of labels on DNA and histone octamer can be the same or different. The positioning of the labels in this scheme can be determined using a crystallographic model of nucleosome for a known nucleosomal DNA sequence. Examples of fluorescent labels placed on both nucleosome-forming DNA and histone are detailed by Li et al. (2004) and (2005), supra. Pursuant to the latter report, the nucleosome-forming DNA is labeled with a single donor Cy3 label, while two symmetry related acceptor dyes Cy5 are attached to histones H3. Pursuant to Li et al. (2004), the nucleosome-forming DNA is labeled with a single Cy3 label, while four Cy5 labels are placed to H3 and H2 histones.

### Labels on Histone Proteins

In some embodiments, the biosensor can have labels associated only with histone proteins while nucleosome forming DNA remains unlabeled. Pursuant to this labeling scheme, one label can be placed, for example, on one of the proteins of the H3/H4 tetramer and the other label on a H2A/H2B dimer to follow events of dissociation of the H2A/H2B dimer. Other labeling arrangements can be determined using crystallographic models of nucleosomes.

### Multiple Donor-Acceptor

In some embodiments, the biosensor can comprise more than one donor-acceptor pair of labels. For example, dye pairs Cy3/Cy5 and Cy5.5/Cy7 can be placed on the nucleosome. Such multipair labeling can allow following dynamic behavior of more than one distance in the nucleosome.

### Non Donor-Acceptor Fluorescent Labels

In some embodiments, the first and the second labels can be fluorescent labels that do not form a donor-acceptor. Instead, the first and the second labels can be such that they quench or suppress each other's emission signal when they are in a close, interfering proximity. For this embodiment, the first and second labels can be labels that have similar excitation/emission characteristics. Preferably, the first and second labels have identical excitation/emission characteristics and are labels of the same type, which means that, if the first label is a TAMRA fluorophore, for example, then the second label is a TAMRA fluorophore as well. Measurement of dynamics for DNA labeled with two TAMRA dyes is detailed in Lang, M L, Fordyce, P M and Block, S M, “Combined optical trapping and single-molecule fluorescence,” Journal of Biology, 2: 6 (2003), http://jbiol.com/content/2/1/6.

Measurement of an emission signal for this embodiment involves illuminating the biosensor with a light with a common excitation wavelength for the fluorescent labels, which can be a monochromatic light from a laser source. The biosensor detects the dynamic state of the nucleosome by measuring a fluorescence intensity at a common emission wavelength of the fluorescent labels. Same instrumentation as for the biosensor comprising a donor-acceptor pair can be utilized for measuring fluorescence in this biosensor embodiment.

In the nucleosomal configuration, the first and second labels are in a close, interfering proximity and the emission signal associated with the labels, i.e., an emission signal at a common wavelength of the labels, is suppressed. Unwrapping of the DNA, whether partial or total, will result in an increase of the emission signal associated with the labels.

Same considerations apply for positioning the labels in a close, interfering proximity in the nucleosomal state for this biosensor embodiment as for the embodiment utilizing a donor-acceptor fluorescent labels.

As for fluorescent labels forming a donor acceptor pair, the first and the second labels can be placed (i) on nucleosome forming DNA only; (ii) on both nucleosome forming DNA and histone proteins and (iii) on histone proteins only.

### Metal Nanoparticles

In another embodiment of the invention, the nucleosome dynamic behavior is monitored using DNA labeled with two metal nanoparticles by taking advantage of the strong distance dependence of the nanoparticles plasmon resonance coupling. Metal nanoparticles are generally known, as evidenced, for example, by METAL NANOPARTICLES, Feldheim and Foss (eds.) (Marcel Dekker, 2001). Metal nanoparticles scatter light effectively at their plasmon frequency, which can depend on nanoparticle composition, size, shape, and the dielectric function of the constitutent metal and the surrounding medium, respectively. Due to their scattering ability, metal nanoparticles were suggested as tracer labels in biological applications by Yguerabide et al., Analytical Biochemistry 262: 137-56 & 156-76 (1998), and in U.S. Pat. No. 6,214,560 and No. 6,586,193 to Yguerabide et al.

When two nanoparticles are brought into interfering proximity, which usually is about 2.5 times particle diameter, their plasmons couple in a distance-dependent manner, which results in changes in the scattering spectrum associated with the nanoparticles. The distance dependence of plasmon coupling has been investigated at fixed interparticle distances with different nanoparticles shapes, including spherical, elliptical, cylindrical, trigonal prismatic, and opposing tip-to-tip triangular. For instance, see Maier et al., Appl. Phys. Lett. 81: 1714-16 (2002); Rechberger et al., Opt. Commun. 220: 137-41 (2003); Haynes et al., J. Phys. Chem. B 107: 7337-42 (2003); Wei et al., Nano Lett. 4: 1067-71 (2004); and Fromm et al., Nano Lett. 4: 957-61 (2004).

The distance-dependent plasmon coupling phenomenon has suggested the use of metal nanoparticles in dynamic molecular rulers; i.e., tools for detecting dynamic geometries of molecules labeled with metal nanoparticles based on the scattering wavelengths of the nanoparticles. See Reinhard et al., Nano Lett. 5: 2246-52 (2005), and Sönnichsen et al., Nature Biotechnology 23: 741-45 (2005).

In a similar manner, the present invention takes advantage of the distance dependence of plasmon resonance coupling between metal nanoparticles, thereby to monitor nucleosomal dynamic behavior. In the invention, DNA that can form a nucleosome is labeled with two metal nanoparticles in such a manner that the distance between the nanoparticles is greater when the DNA does not form a nucleosome (i.e., when the nanoparticles are in the first proximity), compared to the case when the DNA wraps around histone subunits to form a nucleosome (nanoparticles are in the second proximity). Thus, a difference is detectable in the scattering signal associated with the nanoparticles in the first proximity and the second proximity, respectively. Monitoring of the nucleosomal dynamic behavior then can be carried out, pursuant to the invention, by measuring plasmon resonance scattering signal associated with the nanoparticles.

In the first proximity, the nanoparticles are separated by at least 1.5 times, preferably at least 2.0 times, and most preferably at least 2.5 times the size (diameter) of the individual nanoparticle. For an arbitrary DNA sequence, establishing positioning of the nanoparticles is an empirical endeavor, affected by the DNA sequence, by nanoparticle composition, size, shape and by the dielectric constant of the surrounding medium. When the DNA is prepared using a known nucleosomal sequence as a template, one can use the crystallographic model or structure of the nucleosomal sequence to select positions of the nanoparticles on the DNA.

The nanoparticles can be attached to the chosen DNA sequence via nanoparticle-binding functionalities on the DNA. Thus, the DNA can be prepared by PCR, using a biotinylated primer and a digoxygenin-labeled primer, as described by Tomschik et al. (2005), supra, and Zheng et al. in 19 PROTEIN-PROTEIN INTERACTIONS, A MOLECULAR CLONING MANUAL, 2^{nd} ed. 1-19 (Cold Spring Harbor Laboratory Press). In this case, the biotinylated functionality binds to streptavidin-coated nanoparticle, while the digoxygenin functionality can bind to anti-digoxygenin antibody-coated nanoparticle.

Binding of nanoparticles to the DNA can be verified by known techniques, such as atomic force microscopy and dynamic light scattering.

In some embodiments, the DNA can be immobilized on the surface through one of the nanoparticles. Thus, the first nanoparticle label has a coating that both binds to the DNA and facilitates immobilization of the DNA on a surface (e.g., can adhere to the surface), while the second nanoparticle has a coating that binds to the DNA but does not adhere to the surface. For instance, the first nanoparticle can be coated with streptavidin and the second nanoparticle can be coated with anti-digoxygenin antibodies. In this case, the streptavidin-coated nanoparticle can adhere to a surface treated with biotinylated bovine serum albumin (BSA), while the anti-digoxygenin antibody-coated nanoparticle cannot.

To avoid formation of networks of DNA and nanoparticles, the ratio between binding coating molecules and nanoparticles can be varied during coating. This is done in order that only one or two binding molecules are present on the surface of the nanoparticle.

Suitable metal nanoparticles can be any that exhibit plasmon resonance scattering. Preferably, nanoparticles should (1) possess a high degree of homogeneity, both in size and shape; (2) exhibit a strong single peak scattering signal both as individual, undimerized, unclustered nanoparticles particles and in the dimerized state, i.e., when they are brought together after nucleosome formation; (3) have as large as possible of a wavelength separation between the scattering signal from individual, undimerized, unclustered nanoparticles and from the dimerized nanoparticles; (4) be associated with protocols for binding to the DNA. The nanoparticles can be, for example, gold or silver nanobeads with diameters from approximately 10 nm to approximately 100 nm.

Monitoring the nucleosomal dynamic behavior is carried out by measuring the scattering signal associated with the nanoparticles, which involves exposing the DNA to a polychromatic white light and analyzing scattered light from the DNA, typically using a spectrometer. Measuring scattered signal preferably is carried out in a dark field, for example, by means of dark-field microscopy as described Reinhard et al. (2005), supra, including “Supporting Materials.”

The changes in the nucleosomal dynamic state will be reflected in the measured scattering signal. For totally disassembled nucleosome, the maximum of the scattering signal will have a lower wavelength than that for the completely assembled nucleosome.

### Transcription Factors and Nuclear Receptors

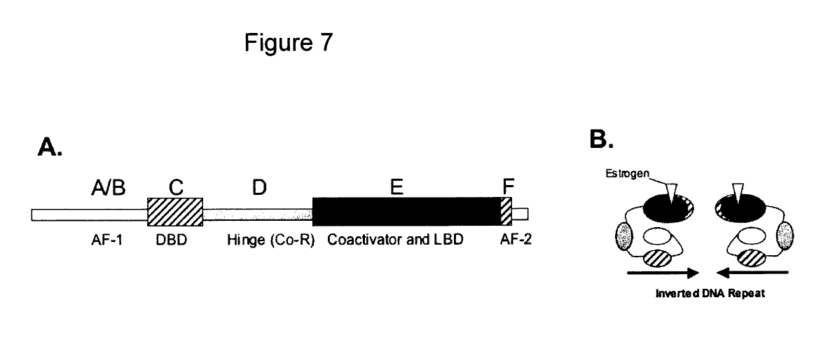

A biosensor of the invention can be applied for detecting events of transcriptional regulation. In eukaryotic cells, transcription is regulated by transcription factors, which are proteins that bind DNA at a specific promoter or enhancer region or site. Accordingly, the nucleosome-forming DNA can include at least one DNA sequence element, involved in transcriptional regulation, that is incorporated either in the core nucleosomal DNA or in the linker DNA. Examples of suitable transcriptional factors and DNA sequences involved in transcriptional regulation can be found, for instance, in Voet and Voet, BIOCHEMISTRY (3rd ed.) 1448-1480 (John Wiley & Sons, 2004). The biosensor of the present invention can be applied in detecting events of transcriptional regulation that involve ligand-inducible transcription factors, such as nuclear receptors. The nuclear receptor superfamily comprises more than 150 proteins that can bind a variety of ligands, such as steroid hormones (glucocorticoids, mineralocorticoids, progesterone, estrogens, and androgens) and thyroid hormones, vitamin D, and retinoids. The nuclear receptors, many of which activate distinct but overlapping sets of genes, are typified by a conserved modular structure that includes, from N- to C-terminus: a poorly conserved transactivation domain containing a ligand-independent activating function AF-1; a highly conserved DNA-binding domain; and a connecting hinge region that contributes to nuclear localization and a ligand-binding domain, which sometimes contributes to nuclear localization and which also harbors a dimerization interface and ligand-dependent activation function (AF-2). The DNA-binding domain contains eight Cys residues, which, in groups of four, tetrahedrally coordinate two Zn²⁺ ions. Some nuclear receptors also contain C-terminal domain (F) of unclear function. FIG. 7A illustrates a conserved modular structure of a nuclear receptor, which includes, from N- to C-terminus (from left to right): region A/B, containing a ligand-independent activating function (AF-1); followed by a DNA-binding domain containing zinc fingers (C); a hinge region (D) that contributes to nuclear localization; a ligand-binding domain (E), which sometimes contributes to nuclear localization and also harbors a dimerization interface; and ligand-dependent activation function (AF-2).

Interacting with a cognate ligand, a nuclear receptor activates a genetic programs by also binding a hormone response DNA element, which comprises single or repeated hexameric DNA motifs that have the sequence 5′-AGGTCA-3′ or a variant thereof. These hexameric sequences can be arranged in the hormone response elements in direct repeats (→n→), inverted repeats (→n←), or everted repeats (←n→), where n represents a 0 to 8 bp spacer. Steroid receptors bind to their hormone response elements as homodimers, whereas other nuclear receptors do so as homodimers, heterodimers, and in few cases, as monodimers. Hormone response elements have been identified even for orphan nuclear receptors, the natural ligands of which are unknown. See, generally, Giguere, Endocrine Reviews 20: 689-725 (1999), and Germain et al., Pure Appl Chem. 75: 1619-64 (2003). FIG. 7B schematically illustrates binding of estrogen receptor to an estrogen receptor DNA element.

### Ligand Action

Ligands binding to nuclear receptors can be classified as agonists and antagonists. Agonists lock the receptor in the active conformation, while antagonists can be viewed as molecules that prevent NRs from adopting this conformation. As nuclear receptors typically have two activation functions, AF-1 and AF-2, a given antagonist may antagonize one or both AFs, and an AF-2 antagonist may act as an AF-1 agonist. A biosensor of the present invention not only can sense binding of ligand to nuclear receptor and binding of the receptor to DNA but also can detect how those binding events modify nucleosome structure. Pursuant to the invention, a ligand that enables nuclear receptor to unwrap DNA is classed as agonists. A ligand is an antagonist if it competes with an agonist, i.e., if it inhibits the promotion of DNA unwrapping by another compound.

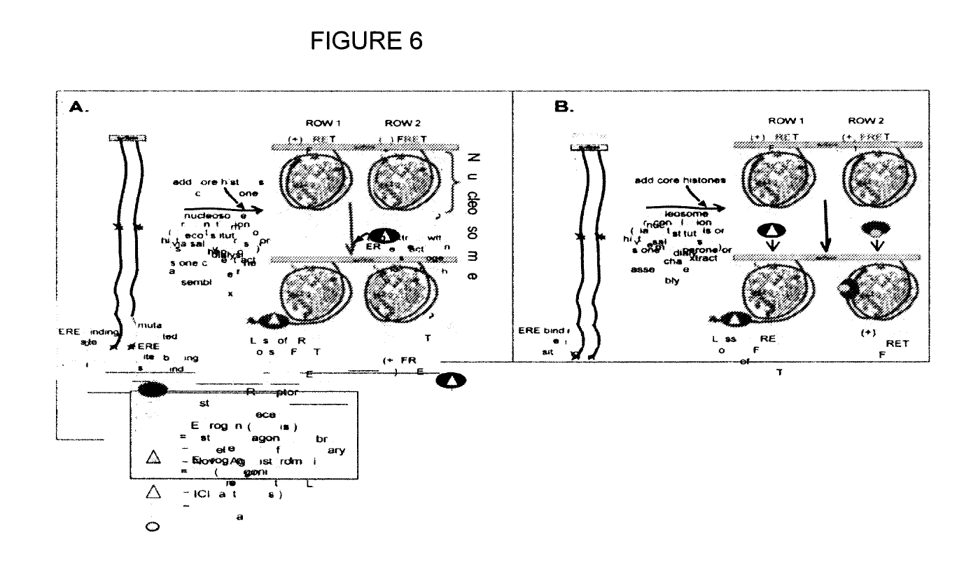

By measuring a change in the emission signal associated with its labels, as described above, a biosensor of the invention can be employed for identifying, in a putative ligand, activity towards a nuclear receptor. In some embodiments, the nuclear receptor can be bound to its nuclear hormone response DNA element, a component of the nucleosome-forming DNA on the biosensor, and the biosensor can be exposed to a putative ligand subsequently. Alternatively, a nuclear receptor unbound to the nuclear hormone response DNA element of the biosensor can be exposed first to a putative ligand, and the biosensor then can be exposed to the nuclear receptor with the putative ligand bound to it (see FIG. 6B).

Exposing the biosensor to a putative agonist ligand will disrupt or modify the nucleosome; accordingly, the labels on the nucleosome will be separated by a greater distance. This effect is manifested in a disappearance or a reduction of the FRET signal for fluorescent donor-acceptor labeling, or in a shift to a higher wavelength of the scattering maximum for labeling with metal nanoparticles.

Identifying a putative antagonist for a given nuclear receptor can be carried out in two essential steps, pursuant to the present invention. The first step entails exposing the biosensor to a putative antagonist, and the second step involves exposing the biosensor thereafter to a saturating amount of a known agonist of the nuclear receptor. An antagonist prevents nucleosome disruption or modification by the known agonist; hence, no changes are observed in the labels-associated emission signal associated or, at least, any changes in the emission signal are less pronounced, compared to the signal occasioned by exposure of the biosensor to the known agonist.

FIG. 6B depicts the identification of a transcriptional activity of a putative ligand. The left panel of FIG. 6B shows two identical nucleosome-forming DNAs, bound to a surface. Each of the DNAs contains an estrogen receptor DNA sequence element (“ERE binding site”) and two fluorescent labels capable of forming a donor-acceptor pair. After histones are added under the nucleosome-forming conditions, each of the DNAs wraps around its respective histone octamer and forms a nucleosome, such that the fluorescent labels for each of the DNAs are in a close proximity and, hence, able to produce a FRET emission signal (see FIG. 6B, upper right panel).

Each of the DNAs is then exposed to an estrogen receptor, to which a putative ligand is bound. Incident to the exposure, the left DNA loses its FRET signal, which indicates that the putative ligand, to which the left DNA was exposed, is an estrogen receptor agonist. The right DNA does lose its FRET signal upon the exposure, indicating that the putative ligand, to which the right DNA was exposed, is not an estrogen receptor agonist. The putative ligand, to which the right DNA was exposed can be an estrogen receptor antagonist or a non-reactive compound.

Some nuclear receptor-binding ligands can have partial agonist/antagonist activity, which may be tissue-dependent. For instance, tamoxifen is an ER antagonist in breast tissue and an agonist in uterine tissue, whereas ER antagonist raloxifene lacks agonist activity in uterus.

To identify whether a putative ligand is active with respect to a nuclear receptor in a specific tissue, a biosensor of the invention can be exposed to the putative ligand in combination with a whole cell or nuclear extract from tissue, the cells of which express the nuclear receptor in question. Alternatively, the biosensor can be exposed to a whole cell or nuclear extract from cells that express the nuclear receptor and that were brought into contact with the putative ligand.

By definition, nuclear extracts contain all or substantially all transcriptional cofactors from source cells but not their nucleosomes and DNA. On the other hand, whole cell extracts contain all or substantially all proteins from source cells, including all or substantially all transcription cofactors. Nuclear extracts can be obtained via conventional methods, as detailed in section 2.1.c.2, infra, and by Dignam et al., Nucleic Acids Res. 11:1475-89 (1983). Accurate transcription initiation by RNA polymerase II in a soluble extract from isolated mammalian nuclei is described by Dignam et al. (1983), while binding of transcription factors present in whole cell extracts is described in Steinman et al., Blood 91: 4531-42 (1998) and R. A. Steinman and A. Iro, Leukemia 13, 54-61 (1999).

### Tissue Specificity of Transcriptional Activators

The biosensor of the present invention also can be employed to identify a tissue specificity in transcriptional factors, particularly transcriptional activators. In this embodiment, the nucleosome-forming DNA of the biosensor comprises, for a transcriptional activator of interest, at least one transcriptional activator response DNA sequence element. Illustrative examples of such elements include: DNA sequence elements, such as C/EBPalpha, that are responsive to transcription factors that promote differentiation, see, e.g. Blobel, Blood 95: 745-55 (2000) and Dixon et al., J. Biol. Chem. 276: 722-28 (2001); DNA sequence elements involved in cellular proliferation, such as AP-1 responsive elements, see Nakabeppu et al., Cell 55: 907-15 (1988), and Takimoto et al., J. Biol. Chem. 264: 8992-99 (1989); and targets of transcription factors that are dysregulated in oncogenesis, such as the beta-catenin partner TCF4 and Stat3, see Kim et al., Oncogene 24: 597-604 (2005), and Turkson et al., Mol. Cell. Biol. 18: 2545-52 (1998).

Pursuant to the invention, a biosensor as described can be exposed to a putative transcriptional activator by way of a whole cell or nuclear extract from tissue in which the transcriptional activator of interest may be expressed. Whether or not the transcriptional activator is functional in the tissue can be determined by measuring a change in the emission signal associated with the labels on the biosensor upon the exposure. That is, the functional transcriptional activator will induce nucleosome modification or disruption, which will be reflected in a change of the emission signal. Illustrative of the transcriptional activators for which a tissue specificity can be determined in this fashion are regulators of tissue development, modulators of cell survival or death signals and sensors of DNA damage.

### Polymorphism of Transcription Regulating DNA Sequence

A biosensor of the invention also can be utilized for determining a functional (or transcriptional) significance of a polymorphism in a DNA regulatory element. For this purpose, a nucleosome-forming DNA in the biosensor can comprise a mutated transcription-regulating DNA sequence element, which differs, by at least one nucleotide, from the non-mutated element for a particular transcription inducer, such as a ligand or a transcription factor. The nucleosome that contains the mutated sequence element then can be exposed to the transcription induce, and a change in emission signal associated with the labels on the nucleosome can be measured. Whether or not the nucleotide change (polymorphism) in the mutated sequence element alters a functionality of the transcription regulating sequence for the inducer can be determined by comparing the measured emission signal change to an emission signal change measured for the non-mutated element; in other words, to an emission signal change measured upon the exposure to the transcription inducer in a biosensor that contains the regular, non-mutated transcription regulating DNA sequence element instead of the mutated sequence element but that, in all other aspects, is identical to the biosensor containing the mutated sequence.

If the change in the emission signal for the biosensor containing the mutated sequence element differs from the change in the emission signal for the otherwise identical biosensor, then the subject nucleotide polymorphism is functionally significant. Conversely, the absence of such difference indicates that the nucleotide polymorphism in not significant functionally. For example, when the biosensor containing the regular non-mutated sequence element loses a FRET signal associated with its fluorescent labels upon the exposure to the transcription inducer and the biosensor containing the mutated sequence element does not lose a FRET signal associated with its fluorescent labels upon the exposure to the same transcription inducer, then the one or more single nucleotide polymorphisms are functionally significant.

FIG. 6A schematically illustrates determination of a functional significance of a polymorphism for estrogen receptor element (ERE). Left panel of FIG. 6A shows two DNAs molecules bound to a surface. Each of the left and right DNAs is labeled with a pair of fluorescent labels forming a donor-acceptor pair. The DNA molecules in FIG. 6A are not in nucleosomal configuration. The fluorescent labels are schematically depicted as stars in FIG. 6A. For each of the left and right DNAs, one of the fluorescent labels is attached at a free, unattached to the surface end of the respective molecule and the other is attached approximately at the center of the molecule. The left DNA contains a regular, non-mutated ERE sequence element (“ERE binding site”) and the right the right DNA contains a mutated ERE sequence element (“mutated ERE binding site”). Otherwise, the left and the right DNAs are identical; that is: (1) the sequence for the left molecule, excluding the ERE binding site, is identical to the sequence for the right molecule, excluding the mutated ERE binding, site, are identical, (2) the fluorescent labels on the left molecule is identical to the fluorescent labels on the right molecule, and (3) a separation along the molecule between the fluorescent labels on the left molecule is the same as a separation along the molecule between fluorescent labels on the right molecule.

Upon adding histones under nucleosome-forming conditions, each of the left and right DNA in FIG. 6A forms a nucleosome (see upper right panel). Each of the left and right DNAs wraps around its respective histone octamer, whereby the fluorescent labels for each of the left and right DNAs are in a close proximity and a FRET emission signal is detectable for each of the left and right DNAs.

The transcription inducer in FIG. 6A is an “activated” estrogen receptor, i.e., one to which estrogen is bound. Upon exposure to the activated estrogen receptor, the nucleosomes formed by the left DNA and the right DNA behave differently: the nucleosome formed by the left DNA loses its FRET signal, while the nucleosome formed by the right DNA does not. The difference in the emission signal change between the left DNA and the right DNA indicates that mutations or polymorphism in the mutated estrogen receptor element are functionally significant. Should there be no difference in the emission signal change, i.e., if the right DNA loses its FRET signal in the same manner as does the left DNA, then the difference or polymorphism in the mutated estrogen receptor element is deemed functionally insignificant.

### High Throughput Format

A biosensor of the invention can be adapted to a high throughput format. Exemplary of this format are a transcription chip or kit that presents a multitude of surfaces, each having one or more nucleosomes associated with it. Each nucleosome contains a nucleosome-forming DNA comprised of at least one transcription regulating DNA sequence element. Each nucleosome is labeled according to one of the labeling arrangements discussed above. The high throughput biosensor system also can include a detector for an emission signal associated with the labels on the nucleosome. Each of the surfaces is in an operational relationship with the detector such that that the emission signal reaches the detector.

The details of the detector and the operational relationship depend on the particularities of the high throughput format. One example is a multiwell plate, of the sort frequently used in fluorescent assays. In this context, the above-mentioned surfaces are represented by the wells on the plate. The standard formats for fluorescent assays include 96-well plate, 384-well plate, and 1536-well plate. The emission signal associated with the labels on the nucleosomes can be detected using a microplate reader available from Molecular Devices Corporation (Sunnyvale, Calif.) or any other such device.

Another example of the high throughput format can be a transcription chip or microarray, which includes a plurality of positionally separated spots on a substrate, each spot having one or more nucleosomes associated with it. Pursuant to this format, the nucleosomes are immobilized on such spots via the methodology described in U.S. Pat. No. 5,972,608 (see, e.g., Example 2). Each nucleosome satisfies the “biosensor” conditions, i.e., the nucleosome-forming DNA of the nucleosome contains at least one transcription regulating DNA sequence element, and the nucleosome is labeled according to one the above-described labeling schemes. For this configuration, the emission signal associated with the labels on the nucleosomes can be detected using, for example, a GeneChip® reader, which is a modified confocal microscope that is available from Affymetrix (Santa Clara, Calif.), or a similar device.

In both microarray and multiwell format, the biosensor system can be used for screening libraries of putative transcriptional inducers, such as putative ligands or putative tissue-specific transcriptional activators. The screening is performed by adding a separate putative inducers to each well or spot and measuring changes in the emission signal from this well or spot.

The microarray format can be also used for determining a transcriptional profile of a tissue. For this application, nucleosome forming DNAs on positionally distinct spots of the microarray will contain transcriptional regulating elements of distinct transcriptional activators. Exposing the microarray to tissue extracts, such as whole cell or nuclear extracts discussed above, will disrupt some nucleosomes of the microarray, while leaving other nucleosomes intact. Measuring emission signal from the spots of the microarray will result in the transcriptional profile of the tissue which will include the data on whether or not a change in emission signal was observed for each spot on the microarray.

Transcriptional profiles can be used for both diagnosis and prognosis of disease conditions. For example, a fingerprint of a given condition can be determined by comparing a transcriptional profile of normal (healthy) tissue and that of a tissue affected by the disease. Transcriptional profiles can be also applied for determining an effect of a stimulus on a tissue by comparing transcriptional profiles of the tissue before and after the stimulus. For instance, the stimulus can be an exposure to a chemical, to radiation, to a virus, or to a hormone.

### Flow Sorting

The high throughput format biosensor system can also be applied for flow sorting. In this embodiment, the system can include a plurality of surfaces, each having one or more one or more nucleosomes associated with it. For flow sorting, the surfaces can be discrete surfaces, separable one from another, such as surfaces of particles that are carried in a flow conduit. The particles can be, for example, microparticles or nanoparticles. Nucleosomes that satisfy the “biosensor” conditions of the present invention can be immobilized on these surfaces.

Thus, the high throughput system in this context would comprise a flow sorter and a emission-signal detector, which can include one or more sensitive and highly responsive photodetector. Preferably, such photodetector can detect single photons of light within the wavelength range of fluorescent labels, e.g. 200 nm-1200 nm, and capable of registering emission signal in millisecond or submillisecond timescale. Illustrative example of the applicable photodetector can be an avalanche photodiode. The particles in the flow are exposed to putative transcription inducers, such as putative ligands for nuclear receptors or putative transcription activators en masse. If the putative transcription inducer disrupts or modifies the nucleosome(s) on the particle, the detector, upon measuring a change in the emission associated with the labels on the nucleosomes, triggers the flow sorter, which would separate particle or particles, for which a change in the emission signal was detected.

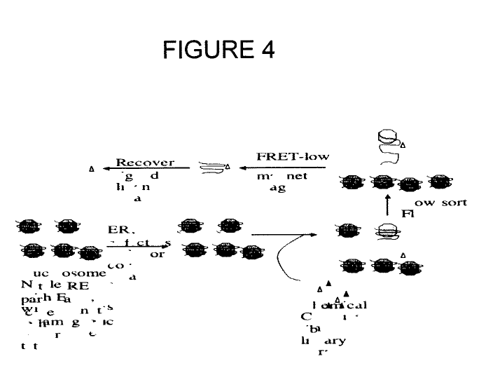

For instance, the flow sorter can be a magnetic flow sorter, and the particles can be particles susceptible to a magnetic field. See FIG. 4, which depicts schematically such a flow sorting biosensor system of the invention. In this instance, the detector triggers a magnetic field change in the flow sorter, which separates the magnetic particle(s) for which a change in the emission signal was detected. Upon the separation, a functional transcriptional inducer, such as a functional agonist ligand or a functional transcriptional activator protein, can be recovered from a surface of a particle, for which a change in emission signal is detected.

The following commentary describes non-limiting illustrations of how the present invention can be applied. In this commentary and in the related “Brief Description of Drawings,” supra, citations to relevant literature are denoted in parenthetical, with reference to the subsequent listing of “Cited Publications.”

Emblematic of the present invention is a study that utilizes DNA-coupled fluorophores to sort, from inactive complexes, transcriptionally active, DNA-binding complexes in nucleosomes, as described above. The priming of nucleosomes for transcription can be followed not only one molecule at a time but also through population averaging, also as described previously.

An advantage of the single-molecule approach is that details about fast processes are obtained including kinetic information that may be lost in asynchronous populations. For instance, rate constants of nucleosomal opening following ligand addition can be calculated for individual nucleosomes. An advantage of the bulk approach is that the differences in agonist activity of different ligands can be quantified on a population basis that controls for variance between individual biosensors.

1. Creating a Nucleosome-Positioning DNA Molecule, Containing an Estrogen Response Element that Transfers Energy as a Function of DNA Assembly into a Nucleosome: Measurement of Single-Pair Fluorescence Resonance Energy Transfer (spFRET)
1.A. Setup of Apparatus to Measure spFRET

FIG. 1A illustrates FRET in schematic terms. FRET occurs if the distance between the two dyes is ˜1 to ˜8 nm. The intensity of FRET varies depending upon the proximity and relative orientation of the two dyes within this range. 50% of the energy transferred between the donor dye and the acceptor dye is at R0, a characteristic distance for each dye pair. The R0 is 6 nm for cyanine Cy3/Cy5 donor/acceptor pair (46). If the molecule undergoes reversible conformational transitions, the intensities of the two dyes will change in an anticorrelated manner with time. In panel A, the intense green signal arises from the donor dye at a >8 nm distance from the acceptor dye. The rise in red fluorescence intensity represents approximation of the two dye positions so that energy is transferred from the donor dye to the acceptor dye. Most spFRET studies use relative changes in the FRET signal to follow dynamics, rather than absolute distance changes (47).

FIGS. 1B and 1C show a scanning confocal fluorescence microscope (SCFM) with millisecond time resolution and a 20:1 signal to noise ratio for single nucleosome visualization that can be used for the present invention.

The cyanine dyes Cy3 and Cy5 (48) are commonly used for FRET, including spFRET. The SCFM utilizes a single-mode fiber to deliver a 532 nm laser beam through a collimating lens and a pinhole, to generate the confocal effect, and a dichroic mirror through the back end of a 60× 1.2 NA water immersion objective to illuminate the sample in a liquid chamber assembled on a glass slide. The glass slide is mounted on a piezo stage that can move 200 microns×200 microns×20 microns in the x-, y-, and z-dimensions with nm precision. The epifluorescence signal is directed through either a Cy3 or a Cy5 filter set and then onto two avalanche photodiodes (APDs in FIG. 1A) for counting of the photon arrival times. In this way, one can register the Cy3 signal and the Cy5 signal simultaneously. Algorithms for analyzing spFRET data are available (5, 49).

1.b. Measuring Real-Time Single Molecule Folding with spFRET

The scanning confocal fluorescence microscope described above can be used to follow real-time folding both of individual DNA molecules and of DNA molecules that are assembled into complexes with proteins such as histones. For example, fast, long-range, reversible conformational fluctuations in nucleosomes between two states: fully folded (closed) with the DNA wrapped around the histone core, or open, with the DNA significantly unraveled from the histone octamer can be observed (49). From this two-state behavior of the nucleosome particle, dwell times were measured for the nucleosome in the closed canonical state or in the open state (i.e. the length of time in one state before a transition to the other state). A histogram of the dwell times in one of these states can be fitted with a single exponential decay function to derive the tau for that state. Tau equals the reciprocal of the rate constant.

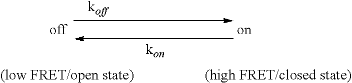

where the k's are first order rate constants, then tau_{off}=1/k_{off} and tauₒₙ=1/kₒₙ.

To exemplify the ability of the SCFM device to track DNA conformation in real-time, according to the invention, a four-way junction DNA substrate is employed, with an acceptor dye, a donor dye, and a biotin each at one of three ends (50). The biotin is used to attach the four-way junction sparsely to a surface that is accessible to a detector, pursuant to the invention, in order to facilitate the investigation of single-molecule dynamics over time. In the presence of magnesium, the four-way junction flips spontaneously between two states, as depicted schematically in the FIG. 2A. With the aforementioned algorithms, the dwell time of DNA in each of the two conformations can be plotted and rate constants for the transition determined (FIG. 2C).

By this approach, doubly-labeled DNA assembled into nucleosomes can be used to study nucleosome dynamics, pursuant to the invention. Thus, it was discovered that single nucleosomes spontaneously transitioned between open and closed states in an equilibrium that was salt-dependent (49). By means of dwell time algorithms mentioned above, the length of time that the nucleosome dwells in the closed state was determined to increase, with salt concentration, from 5 mM to 50 or 250 mM. All SCFM experiments, like the EFFM experiments described below, are performed in liquid cells. These were constructed by drilling inlet and outlet ports on a glass slide. Flow between the ports occurred through a narrow channel constructed between strips of double-stick tape, sealed with epoxy. The glass slide was coated with a polyethylene glycol-streptavidin surface (5). Mononucleosomes assembled with the biotinylated DNA were therefore immobilized when injected into the flow cell for analysis. This setup prevents drying of the sample as well as efficient exchange of buffer.

1.c. Population Analysis of Nucleosome Dynamics

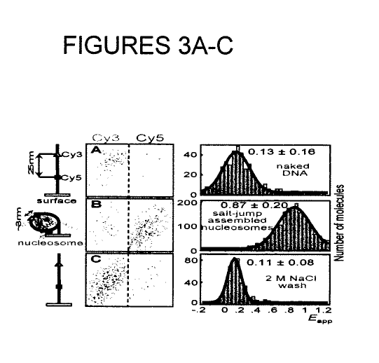

The literature (5, 49) describes the set up a wide-field, intensified charged coupled device-based Evanescent Field Fluorescence Microscope (EFFM) that, in accordance with the present invention, allows for observations of multiple DNA molecules simultaneously. FIG. 3 demonstrates that this system is viable as a sensor of DNA wrapping around histones, in which labeled nucleotides gain or lose contiguity (and therefore FRET) as the DNA wraps or unwraps, as shown schematically at the left of the figure.

FRET transfer is evident upon nucleosome formation in low salt (5 to 250 mM, panel B) and is lost upon nucleosomal disruption at high salt (panel D). The efficiency of energy transfer from Cy3 to Cy5 is measured as Eₐₚₚ. This is calculated roughly as Eₐₚₚ˜I_{acc}/(I_{acc}+I_{don}), where I_{acc} and I_{don} are the intensities of the acceptor and donor dyes, respectively. For more details, see (5).

### 2. Estrogen-Receptor as a Model

For purposes of illustration only, this section focuses on estrogen receptor signaling, as this pathway is well-defined in terms of defined agonists and antagonists and in terms of ER binding to naked DNA and to EREs in chromatin.

Employing a mononucleosome gel shift assay to study ER binding to a 32-bp consensus ERE, Ruh et al. (23) have demonstrated specific binding of ER to the ERE in the presence of either the agonist estrogen or the anti-estrogen H1285. In the present instance, binding was augmented by addition of the high mobility group protein HMGB2.

The results obtained underscore the usefulness of mononucleosome electrophoretic mobility shift assay (EMSA), see Remboutsika et al., J. Biol. Chem. 277: 50318-325 (2002), to validate specific binding of ER to an engineered nucleosomes, in a biosensor according to the present invention. The results also demonstrate that binding to DNA of ligand-loaded receptor does not, on its own, predict whether ligands have agonist or antagonist activity. See also publication 24.

Estrogen signals through two receptors, ERalpha and ERbeta, which overlap in their tissue distribution. These receptors bind, as homodimers or heterodimers, to EREs in the absence of ligand. The two receptors can each bind to and stimulate the same EREs, although they differ in how much they activate transcription driven by specific EREs (25). This may be related to differences in DNA bending induced by each receptor (26) as well as differences in co-activator recruitment (27). Estrogen receptors are primarily nuclear; the subnuclear localization of tagged ERalpha has been reported to be ligand-dependent (28,29). In the setting of chromatin, ERalpha has been demonstrated to be a much more potent transcriptional activator than ERbeta (30). Hereafter, references to “ER” are meant to indicate ERalpha.

2.1. Establish the Ability of Fluorophore-Tagged DNA Containing Estrogen-Response Elements (EREs) to Exhibit Fret when Assembled into Nucleosomes

Illustrative of the routine experimentation associated with the inventive methodology, one can establish optimal spacing of Cy3 and Cy5 tags on ERE-containing DNA, as well as demonstrate FRET-dependence on nucleosomal integrity, by incubation under varying ionic strength.

2.1.a. Cloning of target EREs. To this end, one could use initially a 164-nt ds DNA (49), containing the wild type 35-bp ERE from the vitellogenin promoter (53) (GTCCAAAGTCAGGTCACAGTGACCTGATCAAAGTT), or the wild type 32-bp ERE (TCCAAAGTCAGGTCACAGTGACCTGATCAAAG). A mutant sequence that does not bind to ER (53) can be used in constructs as a control. One could position the ERE 23-bp from the nucleosomal positioning element GTTACAAGGTCTAAACCCGAGTTACAAGGTCTAAACCCGA (54). Ncol-BgIII sites bordering the ERE will facilitate cloning of diverse recognition elements. Alternatively, one also can incorporate the ERE within the GUB sequence (49, 55). Measuring of nucleosomal dynamics for the 32 bp ERE incorporated within the GUB sequence is presented in below, under the section heading “Construction and measurement of spFRET in an ERE containing nucleosome”.

One also can validate the ability of the ERE to bind ER by using, for example, recombinant ER-alpha from Invitrogen, Inc. in electrophoretic mobility shift assays, as detailed in (56). In addition to mutant sequence disclosed in (53), one can prepare other mutant target sequences and use a gel-shift assay to confirm that the prepared mutant sequence does not bind ER. For the 32 bp ERE, such mutant sequence can be TCCAAAGTCAtaTCACAGTGggCTGATCAAAG, where bold lower case letters designate the mutation sites.

Equivalent binding in the absence and presence of agonist (17-beta-estradiol, hereafter “E2”) or antagonist would be expected. The partial agonist/antagonist tamoxifen or the pure antagonist ICI182780 (“ICI”) can be used in this regard (see below). Specific and nonspecific cold competitor sequence can be used to confirm specific binding. Because ERE sequence variation has been shown to modulate ER/co-activator interactions and transcriptional activation (21), one also could clone sequences from the progesterone receptor ERE (PR 1148) and the Xenopus vitamin A2 ERE (EREc38, a perfect 19-bp palindrome) that differ in responsiveness to ER stimulation (21) in cell based assays.

2.1.b Modification of sequence for FRET experiments and assembly into nucleosomes. The 164-bp ERE-containing DNA fragment would be amplified by PCR, such that Cy3 and Cy5 dyes are incorporated into the DNA in positions that abut when the DNA fragment wraps around histones in a nucleosome. One primer would have a 5′-biotin to facilitate immobilization on streptavidin-coated surfaces. Internal aminolink-dC or -dT on the sense and antisense primers will facilitate attachment of fluorophores, as described (49). Positioning of the amino-linkages is directed by the position of the pseudodyad of the nucleosome positioning sequence; experience suggests an insertion of Cy5 at position 47, flanking the ERE, and of Cy3 at position 122.

Core histones could be purified from chicken erythrocytes, for example, as in Tomschik et al. (51), for reconstitution into mononucleosomes. Alternatively, recombinant histones could be prepared with a specific cysteine at various sites for sites for fluorescent labeling for FRET. DNA could be assembled into mononucleosomes via the so-called “salt-jump” method, in which PCR product and carrier DNA are incubated with histone octamers in stepwise dilution of sodium chloride (51). Mononucleosomes then would be separated from unassembled DNA on a 5-25% sucrose density gradient (23).

2.1.c. Confirmation of FRET transfer by ERE-containing mononucleosomes. In an effort to maximize FRET signals in the closed canonical nucleosome, one would seek to position the donor and acceptor dyes within about 5 nm of each other. Modulation of the FRET signal by salt titration or by priming for transcription could then be detected readily. To this end, one initially would ensure that fluorophore locations, relative to the nuclear positioning element, optimizes FRET in the wild type, ERE-containing nucleosomal DNA.
2.1.c.1. Salt sensitivity of FRET signal. Data from the literature (49), reproduced on FIG. 3 and data in FIG. 8, demonstrate a salt dependence of the closed nucleosome conformation, with nucleosomal disruption and loss of FRET at 2M NaCl. By the same token, one could measure changes in FRET signaling as ERE-containing nucleosomes are incubated under varying salt conditions. In particular, changes could be followed over time in a flow cell apparatus previously described (5). The presence of the ERE is not expected to alter salt sensitivity of the nucleosome, and nucleosomal dynamics should parallel reported results (49).

In order to optimize the duration of fluorophore signals prior to bleaching, all reagents preferably would be prepared under nitrogen atmosphere, and reaction solutions preferably would contain a glucose oxidase/catalase system to deplete oxygen. These modifications should counter fluorophore bleaching and extend the observation period (47, 57). One can also add 2 mM Trolox, a Vitamin E-derived antioxidant, that can stabilize Cy5 signal and prevent fluorophore blinking (81).

2.1.c.2. Establishing that Nucleosomes Remain Intact in the Presence of Estrogen Receptor; Use of the Nucleosomal Biosensor to Screen for Ligands Requires that it be not Affected by Unliganded Receptors.

It would be advisable first to confirm that ER binds to the nucleosomal ERE, by radio-labeling the DNA prior to assembly into the nucleosome and then incubating the nucleosome with recombinant estrogen receptor. A supershift of the core histone/DNA (i.e., nucleosome) band by bound ER is expected in EMSA, and further supershift could be tested, using polyclonal anti-ER antibody. Because binding efficacy may be improved with cellular factors, one also could separately add nuclear extract (58), prepared from Chinese Hamster Ovary (CHO) cells, to the ER/mononucleosome mix. The extraction procedure removes CHO cell nucleosomes and DNA while keeping all transcriptional cofactors. (CHO cells do not express either ERα or ERβ and yet contain all necessary cofactors required for ligand-activated ER activity as demonstrated in transfection experiments (59).)

In addition to verifying the formation of ER complexes on ERE-containing nucleosomes, co-immunoprecipitation experiments could be performed. It is expected that pull-down of ER would co-precipitate histones from nucleosomes that contain wild type ERE but would not co-precipitate histones from nucleosomes that contain a mutant ERE that is predicted not to bind ER.

2.1.c.3. FRET and ER binding. For a nucleosomal system of the invention to sense nuclear hormone receptor ligands, it is important that the FRET signal be maintained when the nucleosome is exposed to unliganded (apo-form) receptor. Thus, in order to validate that the DNA remains wrapped around the nucleosome in the presence of apo-form ER, one could preincubate, with recombinant ERα the ERE-containing nucleosome for varying amounts of time with prior to imaging. In this regard, all incubations preferably would be done under conditions that are permissive for ER binding to the nucleosome in gel shift assays, with addition of a glucose oxidase/catalase system to reduce oxygen (e.g. reaction buffer, 12 mM HEPES, pH 7.9, 60 mM KCl, 15% glycerol, catalase, 10 mM DTT, 0.4% (w/v) glucose, 0.1 mg/ml glucose oxidase, and 0.02 mg/ml catalase). One would incubate recombinant ER with Cy3/Cy5-tagged mononucleosomes, in the presence or absence of CHO extract, and confirm that a high FRET signal is maintained; i.e., that the DNA remains wrapped around the histone core and, additionally, that the CHO extract does not perturb the FRET signal.

In addition to demonstrating sustained FRET using recombinant ER, one could confirm that cell extracts expressing transfected ER do not alter the high FRET signal. To this end, for example, CHO cells would be transfected with either wild-type ER or an ER-GFP fusion protein that has been shown to sustain ligand-dependent activation (29). Cells would be depleted of estrogen by 48 hours of culture in phenol red-free medium that contains charcoal-stripped serum, prior to harvest. Expression levels would be validated via Western blotting. In such experiments, extract would be titrated to comparable stoichiometry to recombinant ER.

In this manner, one would confirm that the tested extracts do not alter nucleosomal FRET. Moreover, it could be confirmed, through fluorescence microscopy, that GFP-ER localizes to wild-type (and not mutant) ERE-containing nucleosomes.

2.1.d. Possible Factors Affecting Biosensor Design
2.1d.1. Recombinant ER may not bind to the nucleosome in the absence of cell extracts. Pursuant to the foregoing description, established ERE sequences are employed that have been shown to bind ER in gel shift assays and/or to convey estrogen/ER-dependent transcription to heterologous promoters. Nevertheless, it is possible that recombinant ER alone binds poorly to the sequence in its nucleosomal context. The CHO extracts are expected to support strong binding because cell extracts have reconstituted ER-directed transcription (30). Yet, it is preferable to determine the minimal recombinant system that supports nuclear hormone receptor binding to nucleosomal biosensors.

If recombinant ER alone binds poorly, one could conduct the binding in the presence of HMGB1 or HMGB2, since high mobility group proteins have been shown to bind ER and enhance its binding to nucleosomes (23, 60). This could increase the proportion of informative nucleosomes under study in the wide field. (Based on nucleosome gel shift, all nucleosomes would be loaded with recombinant ER when added with HMGB2; furthermore, HMGB2 should increase binding without triggering nucleosomal remodeling (23).) Another alternative to ensure full nucleosomal loading with ER would entail the use of cell extracts as noted above.

2.1.d.2. Recombinant ER may disrupt FRET in the absence of ligand. It is expected that unliganded ER will bind to the ERE but will not alter FRET, since it should not disrupt the nucleosome. The latter expectation is informed by the DNase studies of Ruh et al. (23), footprinting by Kraus et al. (24), and micrococcal nuclease studies by Metivier et al. (35) in which unliganded (apo-) ER did not remodel the ERE-containing nucleosome, whereas ligand-bound ER did (3).

In a given instance, however, these measures of nucleosomal disruption may be less sensitive than the FRET approach of the present invention, as described above. Thus, ER binding alone conceivably could cause some minor shifts in DNA wrapping that decrease FRET efficiency. If a partial falloff in FRET occurs with unliganded ER and/or ER-containing cell extracts, one can reposition the fluorophores by 3-4 base pairs, relative to each other, and then ascertain whether this enhances the FRET signal in the ER-bound nucleosome.

2.1.d.3 FRET signal may be lost due to non-specific factors. The ERE can be positioned at the 5′ edge of nucleosomal DNA, and the Cy5 acceptor dye can be positioned at the 1^{st} 5′ nucleotide, because DNA at this distal position can be displaced from histones with modest energetic costs and is more accessible to transcription factors than DNA near the central nucleosomal pseudodyad (34, 82-84). Such positioning may select for sensitivity over specificity, however. If assembled nucleosomes are primarily in the open conformation or if CHO extract alone causes FRET loss, one can move fluorophores to internal positions of 47 and 122, where they flank the pseudodyad so that the Cy5 dye can be more stably anchored. The ERE can be also moved one helical turn inwards, to position 13-45.

DNases or proteases may possibly undermine nucleosome activity. To eliminate such possibility, nuclease-free or DEPC treated-water can be used in reagents and protease inhibitors.

### 2.2. Measure Nucleosomal Biosensor Fret Signaling as a Function of ER Ligand Concentration

In accordance with the present invention, adding an ER agonist will destabilize nucleosomes containing wild type ERE in the assembled DNA, and this effect will be manifested by loss of FRET and will be specific to ER as the nuclear receptor added, to agonist (as opposed to antagonist) ligands, and to the integrity of the ERE sequence. As noted above, this nucleosomal activation model could characterized first through the use of cell extracts and, subsequently, in a reconstituted system that utilizes recombinant ER and cofactors, specifically HMGB1 and the SWI/SNF protein Brg1. In that context, changes in FRET would be amenable to examination, pursuant to the invention, at the single-nucleosome level through scanning confocal fluorescence microscopy and at the population level through EFFM. Metivier et al. (35) demonstrated rapid binding of ligand-bound ER to nucleosomal EREs, followed by association of the Brg1 remodeling protein. But the Metivier approach addressed the pooled-population level only, using ChIP, and so it was insensitive to the kinetics of activation of individual nucleosomes (3). Also, a nucleosomal biosensor of the present invention is efficacious in distinguishing agonists from antagonists and in distinguishing extracts from cell types that are permissive or resistant to agonist function.

2.2.1. Do extracts from ligand-exposed cells decrease biosensor FRET relative to ligand-depleted cells; i.e., does FRET loss signal the start of transcriptional activation?
2.2.1.a. Nuclear extracts, prepared from ER-expressing cells exposed to E2 (agonist), ICI (antagonist) or nonligand (cholesterol or Vitamin D3), would be tested for an ability to remodel ERE-containing nucleosomes, i.e., to disrupt FRET

CHO cells would be transfected with an ERα expression plasmid for 16 hours and would be depleted of estrogen by 48-hour culture in phenol red-free Eagle's MEM medium, containing 5% charcoal-stripped calf serum. Cells then would be exposed for six hours to 10 nM E2, ICI, cholesterol or Vitamin D3, and nuclear extracts would be harvested. (ER is predominantly nuclear in all cases (29).) Equivalent ER in extracts would be validated by Western Blotting. Experiments with co-transfected ERE-luciferase reporter constructs also can be conducted to verify E2-specific transcriptional activation, as detailed in (85)-(88).

An additional negative control could include CHO cells transfected with empty vector or with a 46-kD ER mutant, ER-46 (35), which does not transactivate. These control transfectants should lack effect in both the presence and absence of E2.

These extracts would be tested, pursuant to the invention, for their ability specifically to remodel nucleosomes that contain wild-type EREs. There is an expectation of significant remodeling (i.e., FRET loss) in evidence only with extracts from wild-type, ER-expressing, E2-exposed cells. The extracts also could be tested against mutant EREs, which are expected not to respond to ER and ligand. Incubation and analysis would be performed as follows:

Nucleosomes (7 nM solution) are injected into the flow cell, affixed and washed and preincubated in reaction buffer (see 2.1.c.). Fluorescence of the immobilized nucleosomes are acquired by EFFM microscopy in ten 100-msec images, which are acquired successively and then time-averaged. This will localize the position of all nucleosomes that are loaded with coiled ERE-containing DNA (i.e., that exhibit high FRET). At this point, the input valve is opened, to enable preloaded cell extract to flow into the chamber. The shutter is opened, either immediately or at variable time points after mixing, and images are acquired continuously, to inform real-time determination of changes in FRET levels of the aforementioned nucleosomes.

2.2.1.b. Kinetic analysis of ligand-mediated remodeling. With the present invention it is possible to study the effect, in real-time, of ligand addition on nucleosomes that have been preloaded with ER. This would be done, for instance, by incubating nucleosomes with cell extracts from CHO cells, transfected with wild type ER, that were cultured in phenol-red free medium with stripped serum. (These are extracts that, per 2.2.1.a. above, should not remodel nucleosomes because of the lack of agonist.)

Addition of E2 agonist but not ICI antagonist is expected to disrupt FRET. After appropriate incubation to establish equilibrium binding of ER to the nucleosome, the nucleosome field would be imaged for one second, as above, to localize high FRET nucleosomes via EFFM. At this point either E2 or ICI would be added, up to a concentration of about 10 nM, and images would be acquired immediately, thereby to detect real-time shifts in FRET. FRET loss is expected with E2 addition. As a negative control, extracts of cells transfected with the nonfunctional ER mutant ER-46 will be preloaded onto nucleosomes in a parallel experiment. The kinetics of E2 binding to ER (Kₐ 1.0×10⁶ M⁻¹ s⁻¹ (61)) and co-activator assembly [t_{1/2}˜2 seconds (62)] suggest that changes in FRET should be readily discerned within the timeframe of the experiment prior to dye photobleaching.

Pursuant to the invention, high resolution kinetics of ligand-mediated remodeling would be determined at the individual nucleosome level, using a SCFM instrument as described above. This would enable one to calculate Tao values for the dwell time in the high FRET state for vehicle control, E2, and ICI, respectively.

To determine whether nucleosomes are remodeled irreversibly by receptor/ligand complexes, one could add a 100-fold excess of ERE fragment to the inventive nucleosome biosensor, either immediately after addition of cell extract (2.2.1.a), concurrently with ligand (2.2.1.b), or at later time points, as a function of the kinetics. Resumption of FRET after addition of excess ERE (but not excess mutant ERE) would confirm that ongoing ER binding at its recognition site was necessary for the nucleosome to stay in the open, activated conformation, and that the effects were not due to artefactual nucleosomal opening.

In such experiments, EFFM (wide field) instrument imaging preferably would be undertaken first, in order to ascertain the magnitude of the effect in a pooled nucleosomal population (see FIG. 3). Single molecule kinetics then would be established, using SCFM instrumentation as in FIG. 2. See also publication 49.

2.2.1.c. Tissue specificity of nucleosomal stimulation. Tamoxifen is an antagonist of ER signaling in breast but an agonist in uterus. E2 is an agonist in both tissues. In order to test whether this relationship can be reproduced, using a nucleosomal biosensor of the invention, one could conduct experiments comparable to 2.2.1.b., supra, using extracts from breast cancer cells (MCF-7) or endometrial cancer cells [Hec-1, (63)] that have been ligand-starved prior to harvest. In each case, nucleosomes would be preincubated with extract and imaged with EFFM, and either E2 or tamoxifen would be added for real-time imaging. Tamoxifen would be expected to disrupt FRET in the Hec-1 extract (i.e., to act as an agonist) but not in the MCF7 extract, whereas E2 will disrupt FRET in both cases. By the same token, this versatility of the biosensor, as a functional readout, could be utilized with extracts of rare or hard-to-transfect tissues that are incompatible with reporter-based functional assays.
2.2.2. Nucleosomal remodeling by a nuclear hormone receptor, as evidenced by FRET loss, reconstituted in a cell-free system. In vitro reconstitution of ligand-dependent remodeling would facilitate a carefully controlled dissection of NHR-mediated activation at the population and single-molecule level. It also would enable stepwise addition of factors, in order to dissect the triggering event for nucleosomal activation detected by the inventive biosensor.
2.2.2a. Incubation of the nucleosomal biosensor with recombinant ER and ligand in the absence of extracts. As noted in 2.1, ERE-containing DNA will be assembled in nucleosomes so that the FRET signal will be maintained in the presence of ER. The nucleosomes would be bound and imaged in solid state on a streptavidin grid, as in 2.1. Preincubation of nucleosomes would occur in the flow cell with ER, as in 2.2.1b, possibly with equimolar HMGB1 if indicated in nucleosomal gel shift experiments. The ER-bound nucleosomes then would be exposed to E2 at concentrations of 1 fM-10 nM, and imaging of FRET fluorescence would occur either immediately or after a delay. In a parallel analysis, fluorescently-labeled BSA-estrogen would be added and its localization on nucleosomes confirmed through fluorescence microscopy (64). The kinetics of binding of this conjugate are slower than unconjugated estrogen and would define an outside time limit for ligand association to occur. An alternative approach could entail preincubating ER and estrogen, and then adding this complex to the nucleosomes.
2.2.2b. Components of an in vitro remodeling system. To reconstitute a ligand-dependent, ER-driven chromatin remodeling system, using recombinant and/or purified proteins, would involve an analysis of protein requirements for ER-driven transcription from chromatin. One such requirement is for a multicomponent “Mediator” complex, which interacts through the TRAP220 protein with ER (65). This Mediator complex has been shown to interact synergistically with p300/SRC (steroid receptor co-activator) to drive estrogen-induced transcription (66). Biosensor activity, according to the present invention, does not require all of the machinery involved in successful transcriptional activation, however. Rather, it only requires the involvement of protein complexes that will trigger initial steps in that cascade, i.e., to promote partial unwrapping of the (FRET-signaling) DNA from the histone core.

SWI/SNF complexes, particularly the protein Brg1 and its cofactors, have been shown to mediate chromatin remodeling by nuclear receptors in vivo (67). This complex binds directly to ligand-activated ER (68, 3). Because the Brg1 chromatin-remodeling complex is a general mediator of chromatin remodeling, it may suffice to modulate FRET signaling in the presence of liganded ER.

Kinetic analysis of proteins recruited by ER during transcription has demonstrated that Brg1 binds the promoter, immediately after initial ER/estrogen binding, followed by the methyltransferase PRMT1 and the histone acetyltransferase p300. It is worthwhile to test whether the addition of any or all of these proteins will reconstitute the ability to detect ligand-directed changes in FRET.

In this regard, it is notable that the catalytic SWI/SNF subunit Brg1 can remodel nucleosomes on its own (69), an activity augmented by non-catalytic components BAF155 and BAF170. Estradiol-directed remodeling could be controlled by BAF57, a non-catalytic protein that binds to ligand-activated ER, to Brg1, and to the co-activator protein SRC1 (70).

To reconstitute specific chromatin remodeling in vitro, one would co-incubate wild type or mutant ERE-containing nucleosomes with E2/ER or ICI/ER as in D2.2a. Equimolar Brg1, obtainable from Jena Biosciences (Dortmund, Germany), would be added and measurement would be made of the FRET loss that is specific to wt ERE-containing nucleosomes in a buffer of 12 mM HEPES, pH 7.9, 60 mM KCl, 15% glycerol, catalase, 10 mM DTT, 2 mM MgCl2, 4 mM ATP, 0.4% (w/v) glucose, 0.1 mg/ml glucose oxidase, and 0.02 mg/ml catalase. In the absence of measurable FRET loss, Flag-tagged BAF57, BAF155 and BAF170 could 1 be added in a 2:1 ratio, relative to Brg1, in keeping with observations in publication 69. Ligand-dependent measurement of FRET loss would be monitored.

The remodeling reaction is expected to be ATP-dependent. This would be tested by ATP deletion in relation to incubation conditions that had decreased FRET. Also, initial validation of reagents would be effected done through measurement of micrococcal nuclease sensitivity of the mononucleosome bound DNA.

It is expected that the combination of estrogen, estrogen receptor, and Brg1 complex will suffice to reconstitute the initial activation signal of agonist-bound ER, and will cause loss of FRET signal. As expected negative controls, one could incubate nucleosomes in the presence of Brg1 complex without ER, or with Brg1 complex plus ER in the absence of estrogen or with Brg1 complex plus ER in the presence of ICI or cholesterol or Vitamin D3 instead of E2.

### Adaptation of Nucleosome Based Biosensor to High-Throughput Format

A 384-well plate format for nucleosomal biosensors of the invention would be compatible for high throughput analysis of ligand, either in small pools that are subsequently deconvoluted (74-76) or in library screens, in which a separate ligand is added to each well. The plates are compatible with a fluorescence plate reader, illustrated by the reader marketed by Molecular Devices Corporation (Sunnyvale, Calif.) or by a SpectroMax M5 multidetection plate reader with Synchromax robotic plate handlers, which can acquire signal from the plane at the base of wells and, hence, is suited for detection of fluorescence from nucleosomes affixed to the bottom surface. Both the Molecular Devices and the SpectroMax M5 readers have very fast acquisition, permitting real-time changes to be followed. Other factors warranting consideration in this regard an adequate biosensor signal for detection, standardization of biosensor plating per well, and clear differences in signal on a population basis, to discriminate agonist from antagonist ligands.

Plating of nucleosomes. In accordance with the invention, biotin-tagged and unlabelled DNA, containing the ERE and nucleosomal positioning element, would be applied at subsaturating concentrations to blocked, streptavidin-coated 384 well plates, of the sort marketed by Pierce, Inc. (Rockford, Ill.). Core histones would be applied in small volume, at high salt concentration that is stepwise diluted in the wells. Following incubation for nucleosome formation, plates would be washed and DNA-associated histone would be visualized with fluorescent anti-H2A antibody. Variance in signal between wells would be noted. Control wells that lack DNA or contain DNA without a nucleosome positioning sequence would indicate any background binding. Once nucleosomal plating conditions are optimized, a series of wells would be plated with fluorophore-labelled DNA assembled into nucleosomes.
Optimization of signal. Initially, it would be preferable to titrate and affix naked DNA to the wells, in order to establish sensitivity and linearity of detection for the donor and acceptor dyes.
Ligand assays in 384-well plates. To validate a given well format, it would be advisable to repeat in the format the assays that had been performed in the flow cell, as described above. All protein and ligand concentrations would be adapted to the average number of nucleosomes per well. This could be quantitated by immunofluorescence, as discussed above, and by running Western blots on a subset of wells resuspended in Laemmli buffer and normalizing to recombinant H2A, obtainable from Upstate Biology, Inc. (Lake Placid, N.Y.).

ERE-containing nucleosome wells (or control wells lacking ERE, histone, or DNA) would be treated, preferably in triplicate, with cell extract from ER-transfected CHO cells treated with estrogen, tamoxifen, ICI or cholesterol or Vitamin D3. In addition, a subset of wells could be treated with the recombinant protein cocktail (see D2.2, supra) plus E2, tamoxifen, ICI, or cholesterol or Vitamin D3. Correlation of FRET loss with agonist activity then would be determined. Variance between wells containing equivalent extract, ER, and ligand also would be calculated. Furthermore, functional ligand would be titrated to determine sensitivity. In addition, the ability of ICI to compete away FRET changes induced by E2 could be tested. Lastly, one could dilute agonist at different concentrations into non-ligand (cholesterol and/or Vitamin D3) and test the sensitivity and specificity of the inventive biosensor to these agonist-spiked mixtures.

A possible factor to consider is variance in nucleosome assembly within wells. Although uniform coupling of biotinylated DNA to commercial, streptavidin-coated wells is expected, the efficiency of nucleosome formation may vary. The salt-jump methodology, supra, entails addition of histone proteins in 2M NaCl that is gradually diluted. One can resort to 96-well plates if volume constraints on addition or uniform mixing make a 384-well format impractical. If uniformity proves problematic nonetheless, then other nucleosome reconstitution techniques may be employed, such as the use of assembly factors ACF1 and NAP-1 (73).

### Flow Sorting

Inventive biosensors in which DNA is coupled to paramagnetic beads should make it possible to screen libraries en masse, in lieu of allocating each library member to a separate well, for high throughput assay. DNA that has lost FRET fluorescence is expected to retain bound ER and ligand, based on the K_{d} of ligand-ER complexes revealed in GST-ER/ligand binding experiments and based on stability of ER-ligand complexes that are bound to DNA upon supershift in nondenaturing EMSA gels. Such experiments would be performed in solution. To facilitate particle detection during flow sorting, the DNA could be coupled to a 1 μm bead, prior to nucleosome assembly. This can be accomplished by using limited streptavidin coupling of the bead, along with biotin labeling of the 5′ terminus of the DNA strand.

DNA and beads would be mixed in a ratio of about 1:3. Nucleosomes would be generated as described above, and nucleosome DNA complexes would be purified by means of sucrose gradients. The formation of nucleosomes on the beadbound DNA would be confirmed by microscopy.

The resultant presence of a paramagnetic bead at the end of the DNA target enables magnetic isolation of ligand/ER/DNA-bound complexes, after sorting, and recovery of ligand into a small volume for analysis.

Pursuant to the invention, one could expose bead-tagged, ERE-containing nucleosomes separately to agonist, antagonist, or vehicle-exposed MCF-7 cell extract. In this embodiment, beads would be sorted according to high Cy5/low Cy3 (FRET) or loss of FRET. Both sorted concentrations would be concentrated, by means of the paramagnetic beads, and associated ligand then could be identified, e.g., using electrospray ionization-mass spectroscopy. In the particular example under consideration, ICI would be expected to sort with the FRET-expressing population, and estrogen with the non-FRET population.

Use of a recombinant protein system would be preferred to use of cell extracts in this context. A recombinant protein system would facilitate modulation of component stoichiometries. In the instance under consideration, the nucleosomal biosensor would be mixed with an equimolar ligand concentration of estrogen, ICI, tamoxifen, and cholesterol, and the distribution of ligand into sorted pools would be determined. Presumably, cholesterol would not be carried forward through the sort.

Potential factors for consideration in this regard are a residence duration of the ligand/receptor complex that is inadequate for sorting, variance in DNA coupling to beads, and the limits of detection. In addition, even though a receptor/agonist complex may durably alter the FRET signal of the nucleosomal biosensor, it may not remain bound to the nucleosome throughout a ligand isolation procedure. This is a particular concern when cell line extracts are used in which transcription complexes could displace bound ER over time (see 3).

In the absence of a neighboring nucleosome with a TATA box, transcriptional progression is unlikely to be complete. Nevertheless, ER may be dislodged from the nucleosome during penultimate transcriptional steps. To minimize this risk, an alternative approach would involve including ADP in the reaction mix, to slow remodeling. Another alternative would be to conduct experiments in the presence of 5,6-dichlororibofuranosylbenzimidazole (DRB), which prevents polymerase-mediated clearance of ER from the nucleosome (3).

In the event that more than one DNA/nucleosome is coupled to a bead, it could damp shifts in the Cy5/Cy3 ratio if only one of the nucleosomes is bound to an agonist, confounding flow interpretation. One can minimize this risk through limited streptavidin conjugation and use of excess beads. DNA and beads would be mixed rapidly, to minimize local concentration spikes. If microscopic analysis of initial conjugates show a high percentage of multiple DNAs/bead, then the mixing ratio can be increased to 10 beads/DNA.

Construction and Measurement of spFRET in an ERE-Containing Nucleosome.

A biotin-tagged DNA construct that substitutes a 32-bp promoter element containing a wild-type estrogen response element (ERE) at the 5′ end of a 147-bp nucleosome-forming DNA (FIG. 8a) was prepared. This DNA contained the GUB sequence that was previously used to position DNA on nucleosomes and was found to comprise a useful positioning sequence for a study of nucleosomal opening by FRET (49, 55). Specific sequences, such as GUB have been found to direct the ordered formation of nucleosomes centered on the pseudodyad position, which is the midpoint of the DNA sequence that orients the histone core. The nucleosome-forming DNA was generated via PCR using a common 90-bp antisense primer that was biotinylated at the 5′ terminus and contained a TAMRA fluorophore at position 76. The TAMRA fluorophore is positioned one base-pair away from the central nucleosome dyad position and is expected to remain fixed in position relative to core histones in the presence of transcriptional activators (77, 78). The sense PCR primer was be a 42-bp ERE-containing oligonucleotide tagged with Cy5 at the 5′ terminus; oligomers were used to amplify GUB template DNA. The TAMRA/Cy5 fluorophore pair is highly effective in spFRET energy transfer (79, 80).

The three-dimensional structure of this DNA assembled into nucleosomes was modeled, and a distance of 3.4 nm between the TAMRA donor and Cy5 acceptor fluorophores was calculated for the assembled nucleosome (see FIG. 8B). Such a distance is well within the Förster radius for FRET to occur.

The resultant ERE-containing DNA was assembled into nucleosomes. In 250 mM salt, the ERE-containing DNA exhibited anticorrelated TAMRA/Cy5 behavior, consistent with FRET energy transfer (FIG. 8C). The ERE-containing, nucleosome-forming DNA construct exhibited FRET, consistent with a nucleosomal state, for up to 30 seconds.

### CITED PUBLICATIONS

1. Sharif M, Privalsky M L. Cell 1991; 66(5):885-93.
2. de The H, Lavau C, Marchio A, Chomienne C, Degos L, Dejean A. Cell 1991; 66(4):675-84.
3. Metivier R, Penot G, Hubner M R, et al. Cell 2003; 115(6):751-63.
4. Laganiere J, Tremblay G B, Dufour C R, Giroux S, Rousseau F, Giguere V. J Biol Chem 2004; 279(18):18504-10.
5. Zheng H, Tomschik M, Zlatanova J, Leuba S. Evanescent field fluorescence microscopy for analysis of protein/DNA interactions at the single-molecule level. In: Golemis E, Adams P, eds. Protein-Protein Interactions, A Molecular Cloning Manual. 2nd ed. Cold Spring Harbor: Cold Spring Harbor Laboratory Press; 2005:1-16.
6. Osborne C K, Bardou V, Hopp T A, et al. J Natl Cancer Inst 2003; 95(5):353-61.
7. Okuno M, Kojima S, Matsushima-Nishiwaki R, et al. Curr Cancer Drug Targets 2004; 4(3):285-98.
8. Jordan V C, Gapstur S, Morrow M. J Natl Cancer Inst 2001; 93(19):1449-57.
9. Wang T, Xu J, Yu X, Yang R, Han Z C. Crit Rev Oncol Hematol 2005.
10. Beer T M, Myrthue A. Mol Cancer Ther 2004; 3(3):373-81.
11. Hershberger P A, Vasquez A C, Kanterewicz B, Land S, Siegfried J M, Nichols M. Cancer Res 2005; 65(4):1598-605.
12. Trump D L, Hershberger P A, Bernardi R J, et al. J Steroid Biochem Mol Biol 2004; 89-90(1-5):519-26.
13. Hershberger P A, McGuire T F, Yu W D, et al. Mol Cancer Ther 2002; 1(10):821-9.
14. Hershberger P A, Yu W D, Modzelewski R A, Rueger R M, Johnson C S, Trump D L. Clin Cancer Res 2001; 7(4):1043-51.
15. Hershberger P A, Modzelewski R A, Shurin Z R, Rueger R M, Trump D L, Johnson C S. Cancer Res 1999; 59(11):2644-9.
16. Grese T A, Sluka J P, Bryant H U, et al. Proc Natl Acad Sci USA 1997; 94(25):14105-10.
17. Chintharlapalli S, Burghardt R, Papineni S, Ramaiah S, Yoon K, Safe S. J Biol Chem 2005; 280(26):24903-14.
18. Germain P, Altucci L, Bourguet W, Rocheltte-Egly C, Gronemeyer H. Pure Appl Chem 2003; 75 (11-12):1619-64.
19. Le Douarin B, Nielsen A L, Garnier J M, et al. Embo J 1996; 15(23):6701-15.
20. Watkins R E, Wisely G B, Moore L B, et al. Science 2001; 292(5525):2329-33.
21. Klinge C M, Jernigan S C, Mattingly K A, Risinger K E, Zhang J. J Mol Endocrinol 2004; 33(2):387-410.
22. Loven M A, Likhite V S, Choi I, Nardulli A M. J Biol Chem 2001; 276(48):45282-8.
23. Ruh M F, Chrivia J C, Cox L K, Ruh T S. Mol Cell Endocrinol 2004; 214(1-2):71-9.
24. Kraus W L, Kadonaga J T. Genes Dev 1998; 12(3):331-42.
25. Loven M A, Wood J R, Nardulli A M. Mol Cell Endocrinol 2001; 181(1-2):151-63.
26. Schultz J R, Loven M A, Melvin V M, Edwards D P, Nardulli A M. J Biol Chem 2002; 277(10):8702-7.
27. Bai Y, Giguere V. Mol Endocrinol 2003; 17(4):589-99.
28. Welshons W V, Lieberman M E, Gorski J. Nature 1984; 307(5953):747-9.
29. Htun H, Holth L T, Walker D, Davie J R, Hager G L. Mol Biol Cell 1999; 10(2):471-86.
30. Cheung E, Schwabish M A, Kraus W L. Embo J 2003; 22(3):600-11.
31. Zlatanova J, Leuba S H. J Mol Biol 2003; 331(1):1-19.
32. Collingwood T N, Urnov F D, Wolffe A P. J Mol Endocrinol 1999; 23(3):255-75.
33. Acevedo M L, Kraus W L. Essays Biochem 2004; 40:73-88.
34. Li G, Widom J. Nat Struct Mol Biol 2004; 11(8):763-9.
35. Metivier R, Penot G, Carmouche R P, et al. Embo J 2004; 23(18):3653-66.
36. Bitsch F, Aichholz R, Kallen J, Geisse S, Fournier B, Schlaeppi J M. Anal Biochem 2003; 323(1):139-49.
37. Potier N, Billas I M, Steinmetz A, et al. Protein Sci 2003; 12(4):725-33.
38. Sun S, Almaden J, Carlson T J, Barker J, Gehring M R. Metab Eng 2005; 7(1):38-44.
39. Cases M, Garcia-Serna R, Hettne K, et al. Curr Top Med Chem 2005; 5(8):763-72.
40. Liao C, Liu B, Shi L, Zhou J, Lu X P. Eur J Med Chem 2005; 40(7):632-40.
41. Schapira M, Abagyan R, Totrov M. J Med Chem 2003; 46(14):3045-59.
42. Brown P J, Smith-Oliver T A, Charifson P S, et al. Chem Biol 1997; 4(12):909-18.
43. Skretas G, Wood D W. J Mol Biol 2005; 349(3):464-74.
44. Beck V, Pfitscher A, Jungbauer A. J Biochem Biophys Methods 2005; 64(1):19-37.
45. Laitinen S, Fontaine C, Fruchart J C, Staels B. Biochimie 2005; 87(1):21-5.
46. Tan E, Wilson T J, Nahas M K, Clegg R M, Lilley D M, Ha T. Proc Natl Acad Sci USA 2003; 100(16):9308-13.
47. Ha T. Single-molecule fluorescence resonance energy transfer. Methods 2001; 25(1):78-86.
48. Mujumdar R B, Ernst L A, Mujumdar S R, Lewis C J, Waggoner A S. Bioconjug Chem 1993; 4(2):105-11.
49. Tomschik M, Zheng H, van Holde K, Zlatanova J, Leuba S H. Proc Natl Acad Sci USA 2005; 102(9):3278-83.
50. McKinney S A, Declais A C, Lilley D M, Ha T. Nat Struct Biol 2003; 10(2):93-7.
51. Tomschik M, Karymov M A, Zlatanova J, Leuba S H. Structure 2001; 9(12):1201-11.
52. Cheng P, Kanterewicz B, Hershberger P A, McCarty K S, Jr., Day B W, Nichols M. Mol Pharmacol 2004; 66(4):970-7.
53. Kumar V, Chambon P. Cell 1988; 55(1):145-56.
54. Wechsler D S, Papoulas O, Dang C V, Kingston R E. Mol Cell Biol 1994; 14(6):4097-107.
55. An W, Zlatanova J, Leuba S H, van Holde K. Biochimie 1999; 81(7):727-32.
56. Steinman R A, Huang J, Yaroslavskiy B, Goff J P, Ball E D, Nguyen A. Blood 1998; 91(12):4531-42.
57. Gurrieri S, Wells K S, Johnson I D, Bustamante C. Anal Biochem 1997; 249(1):44-53.
58. Dignam J D, Lebovitz R M, Roeder R G. Nucleic Acids Res 1983; 11(5):1475-89.
59. McInerney E M, Weis K E, Sun J, Mosselman S, Katzenellenbogen B S. Endocrinology 1998; 139(11):4513-22.
60. Melvin V S, Roemer S C, Churchill M E, Edwards D P. J Biol Chem 2002; 277(28):25115-24.
61. Rich R L, Hoth L R, Geoghegan K F, et al. Kinetic analysis of estrogen receptor/ligand interactions. Proc Natl Acad Sci USA 2002; 99(13):8562-7.
62. Stenoien D L, Nye A C, Mancini M G, et al. Mol Cell Biol 2001; 21(13):4404-12.
63. Jones P S, Parrott E, White I N. J Biol Chem 1999; 274(45):32008-14.
64. Taguchi Y, Koslowski M, Bodenner D L. Nucl Recept 2004; 2(1):5.
65. Kang Y K, Guermah M, Yuan C X, Roeder R G. Proc Natl Acad Sci USA 2002; 99(5):2642-7.
66. Acevedo M L, Kraus W L. Mol Cell Biol 2003; 23(1):335-48.
67. Hsiao P W, Fryer C J, Trotter K W, Wang W, Archer T K. Mol Cell Biol 2003; 23(17):6210-20.
68. Ichinose H, Garnier J M, Chambon P, Losson R. Gene 1997; 188(1):95-100.
69. Phelan M L, Sif S, Narlikar G J, Kingston R E. Mol Cell 1999; 3(2):247-53.
70. Belandia B, Orford R L, Hurst H C, Parker M G. Embo J 2002; 21(15):4094-103.
71. An W, Kim J, Roeder R G. Cell 2004; 117(6):735-48.
72. Vermeulen M, Stunnenberg H G. Biol Proced Online 2004; 6:157-62.
73. Lusser A, Kadonaga J T. Nat Methods 2004; 1(1): 19-26.
74. Swedlow J R and Platani M. Cell Struct. Funct. 2002; 27:335-41.
75. McNally J G, Karpova T, Cooper J., Conchello J A. Methods 1999; 19:373-85.
76. Brown P. J., Smith-Oliver T. A. et al. Chemistry: Biology 4: 909-918, 1997.
77. Boyes J, Omichinski J, Clark D, Pikaart M, Felsenfeld G. Perturbation of nucleosome structure by the erythroid transcription factor GATA-1. J Mol Biol 1998; 279(3):529-44.
78. Zheng B, Georgakis G V, Li Y, et al. Induction of cell cycle arrest and apoptosis by the proteasome inhibitor PS-341 in Hodgkin disease cell lines is independent of inhibitor of nuclear factor-kappaB mutations or activation of the CD30, CD40, and RANK receptors. Clin Cancer Res 2004; 10(9):3207-15.
79. Haustein E, Jahnz M, Schwille P. Triple FRET: a tool for studying long-range molecular interactions. Chemphyschem 2003; 4(7):745-8.
80. Jeong Y J, Levin M K, Patel S S. The DNA-unwinding mechanism of the ring helicase of bacteriophage T7. Proc Natl Acad Sci USA 2004; 101(19):7264-9.
81. Rasnik I, McKinney S A, Ha T. Non-blinking and long-lasting single molecule fluorescence imaging. Nature Methods 2006; November; 3(11):891-3. Epub 2006 Oct. 1.
82. Adams C C, Workman J L. Binding of disparate transcriptional activators to nucleosomal DNA is inherently cooperative. Mol Cell Biol 1995; 15(3):1405-21.
82. McGhee J D, Felsenfeld G. The number of charge-charge interactions stabilizing the ends of nucleosome DNA. Nucleic Acids Res 1980; 8(12):2751-69.
84. Polach K J, Widom J. Mechanism of protein access to specific DNA sequences in chromatin: a dynamic equilibrium model for gene regulation. J Mol Biol 1995; 254(2): 130-49.
85. Boijoux O, Boutonnet C, Giamarchi C, Favre G, Vagner S, Faye J C. Chemical-based translational induction of luciferase expression: an efficient tool for in vivo screening of protein farnesylation inhibitors. Mol Pharmacol 2005; 67(6): 1829-33.
86. Nuttall M E, Stroup G B, Fisher P W, Nadeau D P, Gowen M, Suva L J. Distinct mechanisms of action of selective estrogen receptor modulators in breast and osteoblastic cells. Am J Physiol Cell Physiol 2000; 279(5):C1550-7.
87. Elgort M G, Zou A, Marschke K B, Allegretto E A. Estrogen and estrogen receptor antagonists stimulate transcription from the human retinoic acid receptor-alpha 1 promoter via a novel sequence. Mol Endocrinol 1996; 10(5):477-87.
88. Buteau-Lozano H, Ancelin M, Lardeux B, Milanini J, Perrot-Applanat M. Transcriptional regulation of vascular endothelial growth factor by estradiol and tamoxifen in breast cancer cells: a complex interplay between estrogen receptors alpha and beta. Cancer Res 2002; 62(17):4977-84.

What is claimed is:

1. A biosensor comprising: (A) at least one surface with which at least one nucleosome is associated, each nucleosome thereof comprising a nucleosome-forming DNA that comprises at least one transcription regulating DNA sequence element and that is labeled with a first label and a second label, such that the first label and the second label are (1) in a first proximity when the nucleosome-forming DNA is in nucleosomal configuration and (2) in a second proximity, differing from the first proximity, when the DNA is not in nucleosomal configuration; and (B) a detector for said emission signal correlated with said labels, wherein said surface is in an operative relationship to said detector such that said emission signal reaches said detector.

2. The biosensor of claim 1, wherein said first and second labels are fluorescent labels.

3. The biosensor of claim 2, wherein the first and second labels are fluorescent labels forming a donor-acceptor pair.

4. The biosensor of claim 2, wherein the first and second labels are fluorescent labels of the same type.

5. The biosensor of claim 2, wherein said detector comprises a scanning confocal fluorescence microscope.

6. The biosensor of claim 2, wherein said detector comprises an evanescent field fluorescence microscope.

7. The biosensor of claim 1, wherein said first and second labels are metal nanoparticles.

8. The method of claim 7, wherein said detector is a dark field microscope.

9. The biosensor of claim 1, wherein component (A) comprises a plurality of surfaces, each surface thereof being separated and positionally distinguishable from other surfaces of said plurality, and wherein each said surface is associated, respectively, with nucleosomes that comprise a nucleosome-forming DNA that comprises at least one transcription regulating DNA sequence element and that is labeled with a first label and a second label, such that the first label and the second label are (1) in a first proximity when the nucleosome-forming DNA is in nucleosomal configuration and (2) in a second proximity, differing from the first proximity, when the DNA is not in nucleosomal configuration.

10. The biosensor of claim 9, wherein component (A) comprises a plurality of plates and component (B) comprises a fluorometric imaging plate reader.

11. The biosensor of claim 1, wherein component (A) comprises a conduit through which said nucleosomes pass in a suitable liquid medium and wherein component (B) comprises a flow sorter and one or more avalanche photodiodes adapted to detect said emission signal.

12. The biosensor of claim 1, wherein the transcription regulating DNA sequence element is a nuclear hormone response element.

13. The biosensor of claim 12, wherein said nuclear hormone response element is an estrogen response element.

14. A transcriptional chip comprising (A) a substrate and, attached thereto, (B) a plurality of nucleosomes, each nucleosome thereof comprising a nucleosome-forming DNA that comprises at least one transcription regulating DNA sequence element and that is labeled with a first label and a second label, such that the first label and the second label are (1) in a first proximity when the nucleosome-forming DNA is in nucleosomal configuration and (2) in a second proximity, differing from the first proximity, when the DNA is not in nucleosomal configuration.

15. The transcriptional chip of claim 14, wherein the nucleosomes of said plurality are positionally distinguishable, one from the other, on said substrate.

16. A method of making a transcriptional chip, comprising
(A) disposing on a substrate a plurality of nucleosome-forming DNAs, wherein each DNA of said plurality comprises at least one transcription regulating DNA sequence element and is labeled with a first label and a second label, such that the first label and the second label are (1) in a first proximity when the nucleosome-forming DNA is in nucleosomal configuration and (1) in a second proximity, differing from the first proximity, when the DNA is not in nucleosomal configuration; and
(B) bringing said DNAs into contact with a plurality of core histones under nucleosome-forming conditions.

17. A method for determining activity of putative ligand towards a nuclear receptor, comprising
(A) providing at least one nucleosome comprising a nucleosome-forming DNA that comprises at least one nuclear hormone response DNA sequence element of said nuclear receptor and that is labeled with a first label and a second label, such that the first label and the second label are (1) in a first proximity when the nucleosome-forming DNA is in nucleosomal configuration and (2) in a second proximity, differing from the first proximity, when the DNA is not in nucleosomal configuration;
(B) exposing said nucleosome to at least one putative ligand; and
(C) measuring for a change in emission signal, associated with said labels, that is consequent to said exposing.

18. A method for determining activity of a transcriptional activator in or from a tissue, comprising
(A) providing at least one nucleosome comprising a nucleosome-forming DNA that comprises at least one response DNA sequence element of said transcriptional activator and that is labeled with a first label and a second label, such that the first label and the second label are (1) in a first proximity when the nucleosome-forming DNA is in nucleosomal configuration and (2) in a second proximity, differing from the first proximity, when the DNA is not in nucleosomal configuration;
(B) exposing said nucleosome to a composition comprising said transcriptional activator and extracts from cells of said tissue; and
(C) measuring for a change in emission signal, associated with said labels, that is consequent to said exposing.

19. A biosensor comprising (A) at least one surface with which at least one nucleosome is associated, each nucleosome thereof comprising (1) a nucleosome-forming DNA that is comprised of at least one nuclear hormone response DNA sequence element and that is labeled with at least one first label and (2) a core histone octamer that is labeled with at least one second label, and (B) a detector for an emission signal correlated with said labels, wherein said surface is in an operative relationship to said detector such that said emission signal reaches said detector.

20. The biosensor of claim 19, wherein said first and second labels are fluorescent labels.

21. The biosensor of claim 20, wherein the first and second labels are fluorescent labels forming a donor-acceptor pair.

22. The biosensor of claim 20, wherein the first and second labels are fluorescent labels of the same type.

23. The biosensor of claim 20, wherein said detector comprises a scanning confocal fluorescence microscope or an evanescent field fluorescence microscope.

24. The biosensor of claim 19, wherein said nucleosomes are immobilized on said surface.

25. The biosensor of claim 19, wherein component (A) comprises a plurality of surfaces, each surface thereof being separated and positionally distinguishable from other surfaces of said plurality, and wherein each said surface is associated, respectively, with nucleosomes that comprise (1) a nucleosome-forming DNA that is comprised of at least one nuclear responsive DNA sequence element and that is labeled with at least one first label and (2) a core histone octamer that is labeled with at least one second label.

26. The biosensor of claim 25, wherein component (A) comprises a plurality of plates and component (B) comprises a fluorometric imaging plate reader.

27. The biosensor of claim 19, wherein component (A) comprises a conduit through which said nucleosomes pass in a suitable liquid medium and wherein component (B) comprises a flow sorter and one or more of avalanche photodiodes adapted to detect said emission signal.

28. The biosensor of claim 19, wherein said nuclear hormone response element is an estrogen response element.

29. A transcription chip comprising (A) a substrate and, attached thereto, (B) a plurality of nucleosomes, each comprising (i) a nucleosome-forming DNA that comprises at least one nuclear hormone response DNA sequence element and is labeled with at least one first label and (ii) a core histone octamer that is labeled with at least one second label.

30. The transcription chip of claim 29, wherein the nucleosomes of said plurality are positionally distinguishable, one from the other, on said substrate.

31. A method of making transcription chip, comprising
(A) disposing on a substrate a plurality of nucleosome-forming DNAs, wherein each DNA of said plurality comprises at least one nuclear responsive DNA sequence element and is labeled with at least one first label
(B) bringing said DNAs into contact with a plurality of core histone octamers, wherein each core histone octamer of said plurality is labeled with at least one second label, under nucleosome-forming conditions.

32. A method for determining activity of putative ligand towards a nuclear receptor, comprising
(A) providing at least one nucleosome comprising (1) a nucleosome-forming DNA that comprises at least one nuclear hormone response DNA sequence element for said nuclear receptor and that is labeled with at least one first label and (2) a core histone octamer that is labeled with at least one second label;
(B) exposing said nucleosome to at least one putative ligand; and
(C) measuring for a change in emission signal, associated with said labels, that is consequent to said exposing.

33. A method for determining activity of a transcriptional activator in or from a tissue, comprising
(A) providing at least one nucleosome comprising (1) a nucleosome-forming DNA that comprises at least one response DNA sequence element of said transcriptional activator and that is labeled with at least one first label and (2) a core histone octamer that is labeled with at least one second label;
(B) exposing said nucleosome to a composition comprising said transcriptional activator and extracts from cells of said tissue; and
(C) measuring for a change in emission signal, associated with said labels, that is consequent to said exposing.

34. A method of sorting putative transcription inducers, comprising
(A) providing a plurality of discrete surfaces that are separable, one from another, wherein at least one nucleosome is associated with each surface of said plurality, said nucleosome comprising a nucleosome-forming DNA that comprises at least one transcription regulating DNA sequence element and that is labeled with a first label and a second label, such that the first label and the second label are (1) in a first proximity when the nucleosome-forming DNA is in nucleosomal configuration and (2) in a second proximity, differing from the first proximity, when the DNA is not in nucleosomal configuration;
(B) exposing said discrete surfaces to a putative transcriptional inducer;
(C) measuring for a change in emission signal, associated with said labels, that is consequent to said exposing; and then
(D) separating said discrete surfaces into first surfaces, for which a change in said emission signal is detected, and second surfaces, for which a change is not detected.

35. The method of claim 34, further comprising recovering a functional transcriptional inducer from said first surfaces.

36. A method of sorting putative transcription inducers, comprising
(A) providing a plurality of discrete surfaces that are separable, one from another, wherein at least one nucleosome is associated with each surface of said plurality, said nucleosome comprising (1) a nucleosome-forming DNA that comprises at least one nuclear hormone response DNA sequence element for said nuclear receptor and that is labeled with at least one first label and (2) a core histone octamer that is labeled with at least one second label;
(B) exposing said discrete surfaces to a putative transcriptional inducer;
(C) measuring for a change in emission signal, associated with said labels, that is consequent to said exposing; and then
(D) separating said discrete surfaces into first surfaces, for which a change in said emission signal is detected, and second surfaces, for which a change is not detected.

37. A method of determining a functional significance of a polymorphism in a transcription regulating DNA sequence element of a transcription inducer, said method comprising
(A) providing at least one nucleosome comprising (1) a nucleosome forming DNA that comprises a mutated transcription regulating DNA sequence element and that is labeled with at least one first label and (2) a core histone octamer that is labeled with at least one second label, the mutated transcription regulating DNA regulating sequence element differs from the transcription regulating DNA sequence element in one or more single nucleotide polymorphism;
(B) exposing said nucleosome to the transcription inducer;
(C) measuring for a change in emission signal, associated with the labels, that is consequent to the exposing;
(D) comparing the measured change in emission signal with a change in emission signal associated with the transcription regulating DNA sequence element.

38. A method of determining a functional significance of a polymorphism in a transcription regulating DNA sequence element of a transcription inducer, said method comprising
(A) providing at least one nucleosome comprising a nucleosome forming DNA that comprises a mutated transcription regulating DNA sequence element and that is labeled with a first label and a second label, such that the first label and the second label are (1) in a first proximity when the nucleosome forming DNA is in nucleosomal configuration and (2) in a second proximity, differing from the first proximity, when the nucleosome forming DNA is not in nucleosomal configuration, the mutated transcription regulating DNA regulating sequence element differs from the transcription regulating DNA sequence element in one or more single nucleotide polymorphism;
(B) exposing said nucleosome to the transcription inducer;
(C) measuring for a change in emission signal, associated with the labels, that is consequent to the exposing;
(D) comparing the measured change in emission signal with a change in emission signal associated with the transcription regulating DNA sequence element.
